(19) United States
(12) Patent Application Publication
(10) Pub. No.: US 2026/0253032 A1
(43) Pub. Date: Aug. 27, 2026

(54) SYSTEM

(71) Applicant: SoftBank Group Corp., Tokyo (JP)

(72) Inventor: Chihiro MATSUNAGA, Tokyo (JP)

(73) Assignee: SoftBank Group Corp., Tokyo (JP)

(21) Appl. No.: 19/537,577

(22) Filed: Feb. 12, 2026

(30) Foreign Application Priority Data
Feb. 21, 2025 (JP) 2025-027092

(51) Int. Cl. G06Q 10/10; G06Q 10/40

(52) U.S. Cl. CPC G06Q 10/10; G06Q 10/40

(57) ABSTRACT

The system according to the embodiment comprises a reception unit, an analysis unit, and a proposal unit. The reception unit receives results of health examinations. The analysis unit analyzes the results of health examinations received by the reception unit. The proposal unit proposes appropriate challenges to a user based on an analysis result obtained by the analysis unit.

### CROSS-REFERENCE TO RELATED APPLICATION

The present application claims priority to and incorporates by reference the entire contents of Japanese Patent Application No. 2025-027092 filed in Japan on Feb. 21, 2025.

### BACKGROUND OF THE INVENTION

### 1. Field of the Invention

The technology of this disclosure relates to a system.

### 2. Description of the Related Art

Japanese Patent Application Laid-open No. 2022-180282 discloses a persona chatbot control method executed by at least one processor, comprising: receiving a user utterance, adding the user utterance to a prompt containing instructions related to the character of the chatbot, encoding the prompt, inputting the encoded prompt into a language model, and generating a chatbot utterance in response to the user utterance.

In conventional technology, appropriate challenges have not been sufficiently proposed to users based on the results of health examinations, and there is room for improvement.

### SUMMARY OF THE INVENTION

The system according to the embodiment comprises a reception unit, an analysis unit, and a proposal unit. The reception unit receives results of health examinations. The analysis unit analyzes the results of health examinations received by the reception unit. The proposal unit proposes appropriate challenges to a user based on an analysis result obtained by the analysis unit.

The above and other objects, features, advantages and technical and industrial significance of this invention will be better understood by reading the following detailed description of presently preferred embodiments of the invention, when considered in connection with the accompanying drawings.

### BRIEF DESCRIPTION OF THE DRAWINGS

FIG. 1 is a conceptual diagram showing an example configuration of a data processing system according to the first embodiment;

FIG. 2 is a conceptual diagram showing an example of main functions of a data processing device and a smart device according to the first embodiment;

FIG. 3 is a conceptual diagram showing an example configuration of a data processing system according to the second embodiment;

FIG. 4 is a conceptual diagram showing an example of main functions of a data processing device and smart glasses according to the second embodiment;

FIG. 5 is a conceptual diagram showing an example configuration of a data processing system according to the third embodiment;

FIG. 6 is a conceptual diagram showing an example of main functions of a data processing device and a headset-type terminal according to the third embodiment;

FIG. 7 is a conceptual diagram showing an example configuration of a data processing system according to the fourth embodiment;

FIG. 8 is a conceptual diagram showing an example of main functions of a data processing device and a robot according to the fourth embodiment;

FIG. 9 shows an emotion map where multiple emotions are mapped; and

FIG. 10 shows an emotion map where multiple emotions are mapped.

### DETAILED DESCRIPTION OF THE PREFERRED EMBODIMENTS

Hereinafter, an example of an embodiment of the system related to the technology disclosed herein will be described with reference to the attached drawings.

First, the terminology used in the following description will be explained.

In the following embodiments, a processor denoted by a reference numeral (hereinafter simply referred to as “processor”) may be a single computing device or a combination of multiple computing devices. The processor may be a single type of computing device or a combination of multiple types of computing devices. Examples of computing devices include a CPU (Central Processing Unit), GPU (Graphics Processing Unit), GPGPU (General-Purpose computing on Graphics Processing Units), APU (Accelerated Processing Unit), or TPU (Tensor Processing Unit), among others.

In the following embodiments, a RAM (Random Access Memory) denoted by a reference numeral is a memory where information is temporarily stored and used as a work memory by the processor.

In the following embodiments, a storage denoted by a reference numeral is one or more non-volatile storage devices for storing various programs and parameters. Examples of non-volatile storage devices include flash memory (SSD (Solid State Drive)), magnetic disks (e.g., hard disks), or magnetic tapes, among others.

In the following embodiments, a communication I/F (Interface) denoted by a reference numeral is an interface including a communication processor and an antenna, among others. The communication I/F manages communication between multiple computers. Examples of communication standards applicable to the communication I/F include wireless communication standards such as 5G (5th Generation Mobile Communication System), Wi-Fi (registered trademark), or Bluetooth (registered trademark), among others.

In the following embodiments, “A and/or B” means “at least one of A and B.” In other words, “A and/or B” means it may be only A, only B, or a combination of A and B. Moreover, when expressing three or more items connected by “and/or,” the same concept as “A and/or B” applies.

### First Embodiment

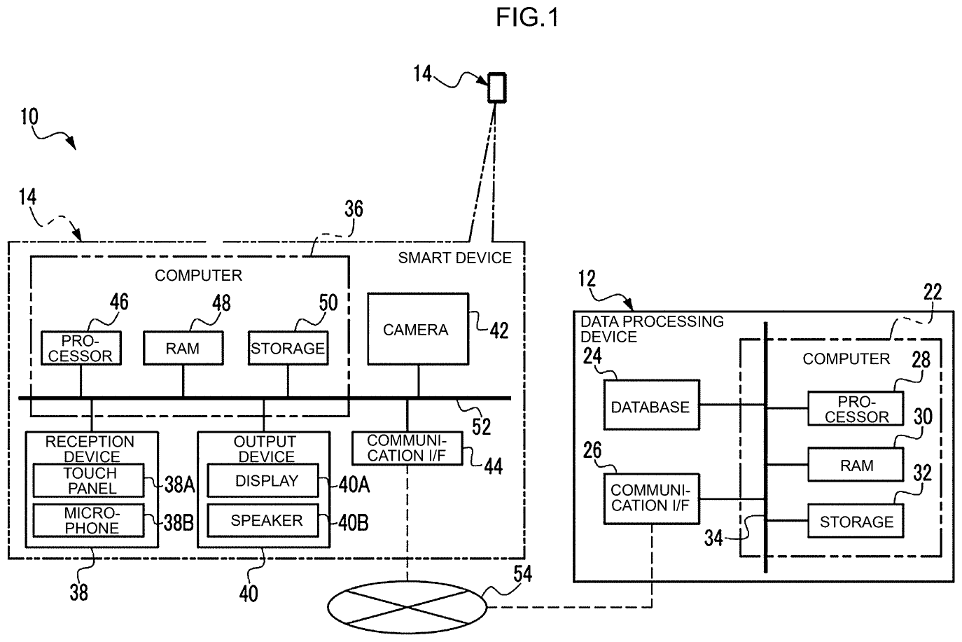

FIG. 1 shows an example configuration of a data processing system 10 according to the first embodiment.

As shown in FIG. 1, the data processing system 10 comprises a data processing device 12 and a smart device 14. An example of the data processing device 12 is a server.

The data processing device 12 comprises a computer 22, a database 24, and a communication I/F 26. The computer 22 comprises a processor 28, RAM 30, and storage 32. The processor 28, RAM 30, and storage 32 are connected to a bus 34. Additionally, the database 24 and communication I/F 26 are also connected to the bus 34. The communication I/F 26 is connected to a network 54. Examples of the network 54 include a WAN (Wide Area Network) and/or a LAN (Local Area Network), among others.

The smart device 14 comprises a computer 36, a reception device 38, an output device 40, a camera 42, and a communication I/F 44. The computer 36 comprises a processor 46, RAM 48, and storage 50. The processor 46, RAM 48, and storage 50 are connected to a bus 52. The reception device 38, output device 40, and camera 42 are also connected to the bus 52.

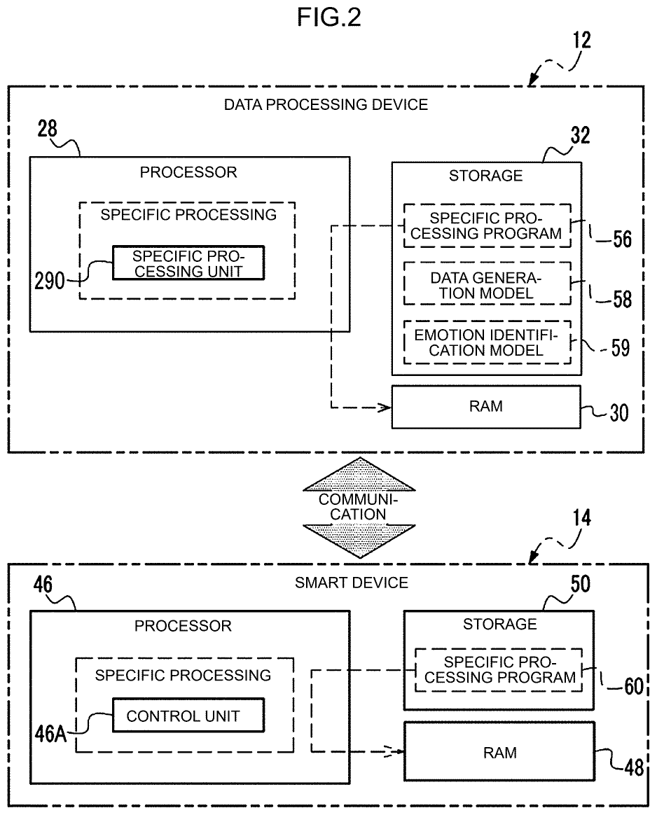

The reception device 38 comprises a touch panel 38A and a microphone 38B, among others, and accepts user input. The touch panel 38A accepts user input by detecting contact from an indicating object (e.g., a pen or finger). The microphone 38B accepts user input by detecting the user's voice. The control unit 46A sends data indicating user input accepted by the touch panel 38A and microphone 38B to the data processing device 12. The data processing device 12 has a specific processing unit 290 (see FIG. 2) that acquires data indicating user input.

The output device 40 comprises a display 40A and a speaker 40B, among others, and presents data to the user by outputting it in a perceptible form (e.g., audio and/or text). The display 40A displays visible information such as text and images according to instructions from the processor 46. The speaker 40B outputs audio according to instructions from the processor 46. The camera 42 is a small digital camera equipped with optical systems such as lenses, apertures, and shutters, as well as imaging elements such as CMOS (Complementary Metal-Oxide-Semiconductor) image sensors or CCD (Charge Coupled Device) image sensors.

The communication I/F 44 is connected to the network 54. The communication I/F 44 and 26 manage the exchange of various information between the processor 46 and the processor 28 via the network 54.

FIG. 2 shows an example of the main functions of the data processing device 12 and the smart device 14.

As shown in FIG. 2, specific processing is performed in the data processing device 12 by the processor 28. The storage 32 stores a specific processing program 56. The specific processing program 56 is an example of a “program” related to the technology disclosed herein. The processor 28 reads the specific processing program 56 from the storage 32 and executes it on the RAM 30. The specific processing is realized by the processor 28 operating as a specific processing unit 290 according to the specific processing program 56 executed on the RAM 30.

The storage 32 stores a data generation model 58 and an emotion identification model 59. The data generation model 58 and emotion identification model 59 are used by the specific processing unit 290. The specific processing unit 290 can estimate the user's emotions using the emotion identification model 59 and perform specific processing using the user's emotions. The emotion estimation function (emotion identification function) using the emotion identification model 59 includes estimating and predicting the user's emotions, but is not limited to such examples. Furthermore, emotion estimation and prediction may include, for example, emotion analysis.

In the smart device 14, specific processing is performed by the processor 46. The storage 50 stores a specific processing program 60. The specific processing program 60 is used in conjunction with the specific processing program 56 by the data processing system 10. The processor 46 reads the specific processing program 60 from the storage 50 and executes it on the RAM 48. The specific processing is realized by the processor 46 operating as a control unit 46A according to the specific processing program 60 executed on the RAM 48. The smart device 14 may also have similar data generation models and emotion identification models as the data generation model 58 and emotion identification model 59, and perform the same processing as the specific processing unit 290 using these models.

Other devices besides the data processing device 12 may have the data generation model 58. For example, a server device (e.g., a generation server) may have the data generation model 58. In this case, the data processing device 12 communicates with the server device having the data generation model 58 to obtain processing results (e.g., prediction results) using the data generation model 58. The data processing device 12 may be a server device or a terminal device owned by the user (e.g., a mobile phone, robot, home appliance, etc.). Next, an example of processing by the data processing system 10 according to the first embodiment will be described.

### Example of the Embodiment

The system according to the embodiment of the present invention is a system that analyzes results of health examinations and proposes challenges such as new sports, physical activities, and travel to the user. In this system, the user inputs health examination results, AI analyzes the results, and proposes appropriate challenges based on the user's physical fitness and health condition. For example, proposals such as “You may be able to walk up to 10 km,” “You can challenge a full marathon,” “You can challenge a 10 km marathon,” and “You can challenge a specific tour during a trip to Machu Picchu” are provided. Through this service, users can find challenges suited to their health condition and enrich their lives. For example, the user inputs health examination results, such as blood pressure, heart rate, body weight, height, and blood test results. These data are input to the AI. Next, the AI analyzes the input data. The AI evaluates the user's physical fitness and health condition and proposes appropriate challenges. For example, the AI evaluates the user's cardiopulmonary function and muscle strength and provides proposals such as “You may be able to walk up to 10 km.” Furthermore, the AI proposes specific challenges based on the user's interests and concerns. For example, if the user is interested in travel, proposals such as “You can challenge a specific tour during a trip to Machu Picchu” are provided. If the user is interested in sports, proposals such as “You can challenge a full marathon” or “You can challenge a 10 km marathon” are provided. Through this service, users can find challenges suited to their health condition and enrich their lives. For example, by learning about their physical fitness from health examination results and challenging new sports based on that, users can enjoy maintaining their health. Additionally, by challenging travel, users can gain new experiences and improve their quality of life. Thus, by analyzing health examination results, the system can propose challenges such as new sports, physical activities, and travel to the user. Specifically, the system receives health examination data input by the user (e.g., blood pressure, heart rate, body weight, height, blood test values, etc., as numerical vectors, each element being a real value, with dimensionality of about 5 to 20) at the reception unit, applies preprocessing such as normalization and missing value imputation, and transfers the data to the analysis unit. The analysis unit evaluates the user's health condition in a multidimensional feature space using large language models such as convolutional neural networks (CNN), multilayer perceptrons (MLP), or Transformer-based models. Examples of AI input include: (1) blood pressure: 120/80, heart rate: 70, body weight: 65 kg, height: 170 cm, blood glucose: 90 mg/dL; (2) blood pressure: 140/90, heart rate: 85, body weight: 80 kg, height: 175 cm, cholesterol: 220 mg/dL; (3) blood pressure: 110/70, heart rate: 60, body weight: 55 kg, height: 160 cm, HbA1c: 5.2%, etc. The AI generates outputs such as physical fitness score (0-100), health risk label (e.g., low, medium, high), and recommended exercise intensity (e.g., mild, moderate, high intensity) based on these input vectors. Output examples include: (1) physical fitness score: 85, health risk: low, recommended exercise intensity: high; (2) physical fitness score: 60, health risk: medium, recommended exercise intensity: moderate; (3) physical fitness score: 40, health risk: high, recommended exercise intensity: mild, etc. These outputs are converted into specific challenge proposals such as “You may be able to walk up to 10 km,” “You can challenge a full marathon,” “You can challenge a 10 km marathon,” and “You can challenge a specific tour during a trip to Machu Picchu” by threshold judgment and rule-based branching in the proposal unit. Furthermore, the proposal unit receives additional input of user interest and concern data (e.g., questionnaire responses, past behavioral history, text data or category labels from SNS posts), vectorizes them using natural language processing models (e.g., BERT or Transformer series models), and performs multi-objective optimization in combination with health condition evaluation results to realize personalized challenge proposals for each user. For AI model training, past health examination data and actual challenge achievement history are used as training data, and weights are optimized using loss functions such as cross-entropy loss and mean squared error. As a result, the system achieves essential improvements in computer technology, not merely automating human tasks, by analyzing vast health examination data and behavioral history in high-dimensional space, surpassing conventional rule-based or manual judgment in accuracy, speed, and personalization. Technical effects include: (1) efficiency of health promotion through optimized challenge proposals for each user; (2) improved proposal accuracy through large-scale data analysis; (3) enhanced user experience through real-time processing; (4) automated history management through database integration; (5) evolution of proposal content through continuous learning of AI models, etc. Specific application fields include health promotion services, corporate health management support, municipal health policies, proposals for sports club members, and health-oriented tour recommendations by travel agencies.

The health examination result analysis system according to the embodiment comprises a reception unit, an analysis unit, and a proposal unit. The reception unit receives results of health examinations. The results of health examinations may include, for example, blood pressure, heart rate, body weight, height, and blood test results, but are not limited to these examples. The reception unit may, for example, store health examination results input by the user in a database. The reception unit can also receive health examination results in real time. For example, the user can input results into the system immediately after undergoing a health examination. The analysis unit analyzes the health examination results received by the reception unit. The analysis unit uses AI to evaluate the user's physical fitness and health condition. For example, the AI evaluates the user's cardiopulmonary function and muscle strength and proposes appropriate challenges. The AI may, for example, use the user's health examination results as input and output evaluation results using an algorithm for evaluating physical fitness and health condition. The proposal unit proposes appropriate challenges to the user based on the analysis result obtained by the analysis unit. The proposal unit uses AI to propose specific challenges based on the user's interests and concerns. For example, the AI uses the user's interests and concerns as input and outputs proposal results using an algorithm for proposing appropriate challenges. The proposal unit may, for example, provide proposals such as “You may be able to walk up to 10 km,” “You can challenge a full marathon,” “You can challenge a 10 km marathon,” and “You can challenge a specific tour during a trip to Machu Picchu.” Thus, the health examination result analysis system according to the embodiment can propose appropriate challenges to the user based on health examination results. Specifically, the health examination result analysis system receives health examination data input by the user at the reception unit (e.g., blood pressure, heart rate, body weight, height, blood test values, etc., as numerical vectors, each element being a real value, with dimensionality of about 5 to 20), and performs preprocessing such as normalization (e.g., Z-score normalization, Min-Max scaling) and missing value imputation (e.g., mean imputation, k-nearest neighbor imputation). The reception unit provides an interface that allows the user to input data from a smartphone or PC terminal immediately after undergoing a health examination, and the input data is stored in the database in real time. The analysis unit inputs the preprocessed data received from the reception unit into AI models such as convolutional neural networks (CNN), multilayer perceptrons (MLP), or Transformer-based large language models. Examples of AI input include: (1) blood pressure: 120/80, heart rate: 70, body weight: 65 kg, height: 170 cm, blood glucose: 90 mg/dL; (2) blood pressure: 140/90, heart rate: 85, body weight: 80 kg, height: 175 cm, cholesterol: 220 mg/dL; (3) blood pressure: 110/70, heart rate: 60, body weight: 55 kg, height: 160 cm, HbA1c: 5.2%, etc. The AI model of the analysis unit processes these input vectors in a multidimensional feature space and generates outputs such as physical fitness score (0-100), health risk label (low, medium, high), and recommended exercise intensity (mild, moderate, high intensity). Output examples include: (1) physical fitness score: 85, health risk: low, recommended exercise intensity: high; (2) physical fitness score: 60, health risk: medium, recommended exercise intensity: moderate; (3) physical fitness score: 40, health risk: high, recommended exercise intensity: mild, etc. These outputs are transferred to the proposal unit, which executes rule-based branching and threshold judgment (e.g., if the physical fitness score is 80 or higher, “You can challenge a full marathon”; if 60-79, “You can challenge a 10 km marathon,” etc.) to generate optimal challenge proposals for the user. Furthermore, the proposal unit vectorizes user interest and concern data (e.g., questionnaire responses, past behavioral history, text data or category labels from SNS posts) using natural language processing models (e.g., BERT or Transformer series models), and performs multi-objective optimization in combination with health condition evaluation results to realize personalized challenge proposals for each user. For AI model training, past health examination data and actual challenge achievement history are used as training data, and weights are optimized using loss functions such as cross-entropy loss and mean squared error. As a result, the system achieves essential improvements in computer technology, not merely automating human tasks, by analyzing vast health examination data and behavioral history in high-dimensional space, surpassing conventional rule-based or manual judgment in accuracy, speed, and personalization. Technical effects include: efficiency of health promotion through optimized challenge proposals for each user, improved proposal accuracy through large-scale data analysis, enhanced user experience through real-time processing, automated history management through database integration, and evolution of proposal content through continuous learning of AI models. Specific application fields include health promotion services, corporate health management support, municipal health policies, proposals for sports club members, and health-oriented tour recommendations by travel agencies.

The reception unit can receive data of blood pressure, heart rate, body weight, height, and blood test results. The reception unit may, for example, receive data of blood pressure, heart rate, body weight, height, and blood test results input by the user. These data are, for example, obtained when the user undergoes a health examination. Blood pressure is measured, for example, using a blood pressure monitor. Heart rate is measured, for example, using a heart rate monitor. Body weight is measured, for example, using a scale. Height is measured, for example, using a stadiometer. Blood test results are measured, for example, using blood test equipment. Thus, the reception unit can receive detailed data of health examinations. Some or all of the above-described processing in the reception unit may be performed using AI or may be performed without using AI. For example, the reception unit may input data entered by the user into AI and have the AI execute the data reception. Specifically, the reception unit provides an interface for receiving health examination data input by the user (e.g., blood pressure, heart rate, body weight, height, blood test values, etc., as numerical vectors, each element being a real value, with dimensionality of about 5 to 20). The reception unit automatically performs format checks (e.g., unit unification, outlier detection), missing value imputation (e.g., imputation using past data or mean values), and normalization processing (e.g., Z-score normalization, Min-Max scaling) on the input data. When using AI, the reception unit inputs the data into an AI model (e.g., autoencoder for outlier detection or unsupervised learning model) and determines the validity of the data and the presence of outliers. Examples of AI input include: (1) blood pressure: 120/80, heart rate: 70, body weight: 65 kg, height: 170 cm, blood glucose: 90 mg/dL; (2) blood pressure: 140/90, heart rate: 85, body weight: 80 kg, height: 175 cm, cholesterol: 220 mg/dL; (3) blood pressure: 110/70, heart rate: 60, body weight: 55 kg, height: 160 cm, HbA1c: 5.2%, etc. The AI model generates outputs such as outlier score (e.g., continuous value from 0 to 1), input data reliability label (e.g., high, medium, low), and automatic imputation candidates (e.g., estimated values for missing data). Output examples include: (1) outlier score: 0.05, reliability: high, no imputation needed; (2) outlier score: 0.35, reliability: medium, imputation candidate: blood glucose 95 mg/dL; (3) outlier score: 0.80, reliability: low, imputation candidate: body weight 60 kg, etc. These outputs are used in subsequent processing of the reception unit, such as determining whether to save data to the database, prompting the user to correct input, or determining whether to transfer data to the analysis unit. When not using AI, similar processing is performed using conventional rule-based processing or simple threshold judgment. Technical effects include that by utilizing AI, the reception unit can greatly improve the efficiency of quality control of input data, outlier detection automation, reduction of input errors, maintenance of database consistency, and real-time input support, thereby enhancing the reliability and user experience of the entire system. Application fields include general health examination data reception operations, electronic medical record input support for medical institutions, corporate health management systems, and municipal health management platforms.

The analysis unit can evaluate the user's physical fitness and health condition. The analysis unit may, for example, evaluate physical fitness and health condition based on the user's health examination results. Evaluation of physical fitness and health condition may include, for example, physical fitness tests and health examination items, but is not limited to these examples. Physical fitness tests may include, for example, cardiopulmonary function tests and muscle strength tests. Cardiopulmonary function tests are performed, for example, using a treadmill. Muscle strength tests are performed, for example, using a dynamometer. Health examination items may include, for example, blood pressure, heart rate, body weight, height, and blood test results. Thus, the analysis unit can evaluate the user's physical fitness and health condition. Some or all of the above-described processing in the analysis unit may be performed using AI or may be performed without using AI. For example, the analysis unit may input the user's health examination results into AI and have the AI execute the evaluation of physical fitness and health condition. Specifically, the analysis unit receives health examination data from the reception unit (e.g., blood pressure, heart rate, body weight, height, blood test values, etc., as numerical vectors, each element being a real value, with dimensionality of about 5 to 20) as input. The analysis unit uses AI models such as convolutional neural networks (CNN), multilayer perceptrons (MLP), or Transformer-based large language models to perform multidimensional feature extraction and evaluate the user's physical fitness and health condition in high-dimensional space. Examples of AI input include: (1) blood pressure: 120/80, heart rate: 70, body weight: 65 kg, height: 170 cm, blood glucose: 90 mg/dL; (2) blood pressure: 140/90, heart rate: 85, body weight: 80 kg, height: 175 cm, cholesterol: 220 mg/dL; (3) blood pressure: 110/70, heart rate: 60, body weight: 55 kg, height: 160 cm, HbA1c: 5.2%, etc. The AI model generates outputs such as physical fitness score (0-100), health risk label (low, medium, high), and recommended exercise intensity (mild, moderate, high intensity) based on these input vectors. Output examples include: (1) physical fitness score: 85, health risk: low, recommended exercise intensity: high; (2) physical fitness score: 60, health risk: medium, recommended exercise intensity: moderate; (3) physical fitness score: 40, health risk: high, recommended exercise intensity: mild, etc. These outputs are transferred to the subsequent proposal unit and used as basic data for challenge proposals to the user. Internal processing of the AI model includes weighted sum operations on input vectors, activation functions (e.g., ReLU, Sigmoid), feature extraction by multiple layers of fully connected and convolutional layers, learning by loss functions (e.g., mean squared error, cross-entropy loss), and optimization by gradient descent of weights. When not using AI, similar evaluation is performed using conventional rule-based processing or simple threshold judgment. Technical effects include that by utilizing AI, the analysis unit can analyze vast health examination data in high-dimensional space and realize high-precision and high-speed recognition of complex health condition patterns and physical fitness evaluation, which was difficult with conventional manual or simple rule-based methods. Application fields include automatic analysis of health examination results, physical fitness evaluation for sports club members, corporate health management support, and municipal health policy evaluation.

The proposal unit can propose specific challenges based on the user's interests and concerns. The proposal unit may, for example, propose specific challenges based on the user's interests and concerns. The user's interests and concerns may be identified, for example, from questionnaires or past behavioral history. Questionnaires may be conducted, for example, in a format where the user answers questions about sports or travel destinations of interest. Past behavioral history may be identified, for example, from records of sports events or travel the user has participated in. Thus, the proposal unit can propose specific challenges based on the user's interests and concerns. Some or all of the above-described processing in the proposal unit may be performed using AI or may be performed without using AI. For example, the proposal unit may input the user's interest and concern data into AI and have the AI execute the proposal of specific challenges. Specifically, the proposal unit receives user interest and concern data (e.g., questionnaire responses, past behavioral history, text data or category labels from SNS posts) from the reception unit or analysis unit and vectorizes them using natural language processing models (e.g., BERT or Transformer series models). Examples of AI input include: (1) questionnaire response: “Sports: running, travel destination: South America”; (2) past behavioral history: “May 2023: participated in 10 km marathon, August 2022: mountain climbing trip”; (3) SNS post: “Recently interested in hiking,” etc. The AI model vectorizes these input data, combines them with health condition evaluation results (e.g., physical fitness score, health risk label), applies multi-objective optimization algorithms (e.g., linear weighting, reinforcement learning-based recommendation models), and generates personalized challenge proposals for each user. Examples of AI output include: (1) proposal: “You may be able to walk up to 10 km”; (2) proposal: “You can challenge a full marathon”; (3) proposal: “You can challenge a specific tour during a trip to Machu Picchu,” etc. These outputs are used for user display screens or notification messages. Internal processing of the AI model includes tokenization of input text, generation of embedding vectors, feature extraction by multiple layers of self-attention mechanisms, integration with health condition evaluation, learning by loss functions (e.g., cross-entropy loss), scoring of recommendation results, and threshold judgment. When not using AI, similar proposals are executed using conventional rule-based processing or simple category matching. Technical effects include that by utilizing AI, the proposal unit can integratively analyze the user's diverse interests and concerns and health condition in high-dimensional space, greatly improving personalization, proposal accuracy, and proposal speed, which was difficult with conventional manual or simple rule-based methods. Application fields include personalized proposals for health promotion services, event recommendations for sports club members, health-oriented tour proposals by travel agencies, and corporate health management support.

The proposal unit can provide proposals such as “able to walk up to 10 km,” “able to challenge a full marathon,” “able to challenge a 10 km marathon,” and “able to challenge a specific tour during a trip to Machu Picchu.” The proposal unit may, for example, provide proposals such as “able to walk up to 10 km,” “able to challenge a full marathon,” “able to challenge a 10 km marathon,” and “able to challenge a specific tour during a trip to Machu Picchu” based on the user's health examination results and interests and concerns. The content and criteria of specific challenges are determined, for example, based on the type of exercise, difficulty level, and achievement goals. Types of exercise may include, for example, walking, running, hiking, etc. Difficulty level is determined, for example, based on exercise intensity or distance. Achievement goals may include, for example, walking a specific distance or running within a specific time. Thus, the proposal unit can propose specific challenges. Some or all of the above-described processing in the proposal unit may be performed using AI or may be performed without using AI. For example, the proposal unit may input the user's health examination results and interest and concern data into AI and have the AI execute the proposal of specific challenges. Specifically, the proposal unit integrates health condition evaluation results received from the analysis unit (e.g., physical fitness score, health risk label, recommended exercise intensity) and user interest and concern data (e.g., questionnaire responses, past behavioral history, text data or category labels from SNS posts), and inputs them into an AI model (e.g., multi-objective optimization recommendation model, reinforcement learning-based proposal model). Examples of AI input include: (1) physical fitness score: 85, health risk: low, recommended exercise intensity: high, interest: running; (2) physical fitness score: 60, health risk: medium, recommended exercise intensity: moderate, interest: travel; (3) physical fitness score: 40, health risk: high, recommended exercise intensity: mild, interest: walking, etc. The AI model generates challenge proposal candidates (e.g., exercise type, difficulty level, achievement goal, reason for recommendation as structured data) based on these input data. Output examples include: (1) proposal: “You can challenge a full marathon,” difficulty: high, goal: complete 42.195 km, reason for recommendation: physical fitness score 85, interest: running; (2) proposal: “You can challenge a specific tour during a trip to Machu Picchu,” difficulty: medium, goal: trekking at an altitude of 2500 m, reason for recommendation: physical fitness score 60, interest: travel; (3) proposal: “You may be able to walk up to 10 km,” difficulty: low, goal: 10 km walking, reason for recommendation: physical fitness score 40, interest: walking, etc. These outputs are used for user display screens or notification messages, allowing users to select challenges according to their health condition and interests. Internal processing of the AI model includes weighting of input features, optimization of multiple objective functions, calculation of recommendation scores, branching of proposal content by threshold judgment, and learning by loss functions (e.g., cross-entropy loss). When not using AI, similar proposals are executed using conventional rule-based processing or simple category matching. Technical effects include that by utilizing AI, the proposal unit can integratively analyze the user's health condition and interests and concerns in high-dimensional space, greatly improving personalization, proposal accuracy, and proposal speed, which was difficult with conventional manual or simple rule-based methods. Application fields include personalized proposals for health promotion services, event recommendations for sports club members, health-oriented tour proposals by travel agencies, and corporate health management support.

The reception unit can estimate the user's emotions and adjust the timing of inputting health examination results based on the estimated emotions of the user. The reception unit may, for example, estimate the user's emotions and adjust the timing of inputting health examination results based on the estimated emotions. Emotion estimation may be performed, for example, using facial recognition, voice analysis, or questionnaire results. Facial recognition may be performed, for example, by analyzing the user's facial expressions captured by a camera. Voice analysis may be performed, for example, by analyzing the tone and speed of the user's voice. Questionnaire results may be estimated, for example, based on the user's answers to questions about emotions. Thus, the reception unit can adjust the timing of inputting health examination results according to the user's emotions. For example, if the user feels stressed, input is prompted during a relaxing time. If the user is busy, input items are narrowed down to allow quick input. Furthermore, if the user is relaxed, detailed input is prompted. Emotion estimation may be realized, for example, using an emotion engine or generative AI with emotion estimation functions. Generative AI may include, for example, text generation AI (e.g., LLM) or multimodal generative AI, but is not limited to these examples. Some or all of the above-described processing in the reception unit may be performed using AI or may be performed without using AI. For example, the reception unit may input the user's emotion data into AI and have the AI execute the adjustment of input timing. Specifically, the reception unit acquires multiple modality data for emotion estimation of the user. For example, for facial recognition, camera images (RGB image tensor, resolution 128×128 pixels, 3 channels); for voice analysis, voice waveform data (1D time series array, sampling rate 16 kHz, length 3-10 seconds); for questionnaire results, text data (free description or selection format, with category labels) are acquired. The reception unit performs preprocessing (e.g., face region extraction, voice noise removal, text normalization) on these data and inputs them into a multimodal AI model (e.g., integrated model of CNN for images, RNN for voice, and Transformer for text). Examples of AI input include: (1) camera image: smiling face, voice: calm tone, questionnaire: “I am relaxed now”; (2) camera image: frowning face, voice: fast and high-pitched, questionnaire: “I am a little irritated”; (3) camera image: expressionless, voice: monotone, questionnaire: “Busy,” etc. The AI model integrates these inputs and generates outputs such as emotion label (e.g., relaxed, stressed, busy), emotion score (continuous value from 0 to 1), and recommended input timing (e.g., immediate, later, nighttime recommended). Output examples include: (1) emotion: relaxed, score: 0.85, recommended timing: immediate; (2) emotion: stressed, score: 0.70, recommended timing: nighttime; (3) emotion: busy, score: 0.90, recommended timing: later, etc. These outputs are used in subsequent processing of the reception unit, such as controlling the display timing of the input screen, automatically narrowing down input items, and sending reminder notifications. Internal processing of the AI model includes feature extraction layers for each modality, concatenation of multidimensional feature vectors in the integration layer, and implementation of multitask learning for emotion classification, score regression, and timing recommendation in the fully connected layer. Training uses actual user input history and emotion-labeled datasets, and weights are optimized using cross-entropy loss and mean squared error as loss functions. When not using AI, similar estimation is performed using threshold judgment of facial feature points, rule-based judgment of voice pitch and speed, and simple matching of questionnaire responses. Technical effects include that by utilizing AI, the reception unit can estimate the user's emotional state with high accuracy and in real time, and automatically adjust the optimal input timing, thereby reducing input stress, improving input completion rate, optimizing user experience, and enhancing overall system efficiency. Application fields include health examination data reception systems, patient input support for medical institutions, corporate health management platforms, and municipal health management services.

The reception unit can analyze the user's past health examination results and select an appropriate input method. The reception unit may, for example, automatically complete input items based on the user's past health examination results. For example, the reception unit may automatically complete current input items based on data previously entered by the user. Furthermore, the reception unit can preferentially propose input methods (such as voice or text) previously used by the user. For example, if the user previously used voice input, voice input is preferentially proposed. Additionally, the reception unit can prompt input at specific times based on the user's past input history. For example, if the user previously entered data at a specific time, input is prompted at that time. Thus, the reception unit can select the optimal input method based on the user's past health examination results. Some or all of the above-described processing in the reception unit may be performed using AI or may be performed without using AI. For example, the reception unit may input the user's past health examination results into AI and have the AI execute the selection of the optimal input method. Specifically, the reception unit acquires, for each user, past health examination data (e.g., blood pressure, heart rate, body weight, height, blood test values, etc., as time series vectors, each element being a real value, with history length of 1 to 10 times), past input method history (e.g., category labels such as voice input, text input, image upload), and input time history (e.g., timestamp, day of week, time zone label) from the database. The reception unit applies preprocessing such as time series feature extraction (e.g., moving average, trend score calculation), category frequency aggregation, and input pattern analysis to these data and inputs them into an AI model (e.g., integrated model of time series RNN, category classification MLP, and recommendation algorithm). Examples of AI input include: (1) past three health examination data, all voice input, input time is weekday night; (2) past five health examination data, mixed text and voice input, input time is weekend morning; (3) past two health examination data, image upload, input time is weekday noon, etc. The AI model generates outputs such as automatic completion candidates for input items (e.g., estimated values for body weight and height), recommended input method (e.g., voice, text, image), and recommended input time (e.g., night, morning, noon) based on these inputs. Output examples include: (1) completion candidate: body weight 65 kg, recommended method: voice, recommended time: night; (2) completion candidate: blood pressure 120/80, recommended method: text, recommended time: morning; (3) completion candidate: cholesterol 200 mg/dL, recommended method: image, recommended time: noon, etc. These outputs are used for input assistance on the reception screen, automatic presentation of input method options, and sending reminder notifications. Internal processing of the AI model includes feature extraction of time series data using LSTM or GRU, embedding vectorization of category data, integration by fully connected layers, recommendation score calculation, and learning by loss functions (e.g., cross-entropy loss, mean squared error). When not using AI, similar processing is performed using simple mean value imputation of past data or rule-based selection of the most frequent input method and time. Technical effects include that by utilizing AI, the reception unit can analyze input tendencies and history for each user in high-dimensional space and automatically propose optimal input methods, timing, and completion values, thereby improving input efficiency, reducing input errors, enhancing user experience, and improving overall system accuracy. Application fields include health examination data reception systems, patient input support for medical institutions, corporate health management platforms, and municipal health management services.

The reception unit can perform filtering based on the user's current lifestyle habits and dietary content when inputting health examination results. The reception unit may, for example, filter health examination results based on the user's current lifestyle habits and dietary content. Information on lifestyle habits and dietary content may be obtained, for example, from meal records, exercise records, and sleep records. Meal records may be obtained, for example, using an application in which the user records daily dietary content. Exercise records may be obtained, for example, using an application in which the user records daily exercise content. Sleep records may be obtained, for example, using an application in which the user records daily sleep patterns. Thus, the reception unit can filter health examination results based on the user's lifestyle habits and dietary content. For example, health examination items related to dietary content are preferentially input based on the user's dietary content. Furthermore, health examination items related to lifestyle habits (such as exercise frequency) are preferentially input based on the user's lifestyle habits. Additionally, health examination items related to sleep patterns are preferentially input based on the user's sleep patterns. Some or all of the above-described processing in the reception unit may be performed using AI or may be performed without using AI. For example, the reception unit may input the user's lifestyle habits and dietary content data into AI and have the AI execute filtering. Specifically, the reception unit automatically acquires dietary content recorded by the user (e.g., table data including food names, quantities, and nutritional information for three meals per day, for one week), exercise records (e.g., time series data of exercise type, intensity, duration, calories burned), and sleep records (e.g., bedtime and wake-up time, sleep duration, sleep efficiency as time series data) from health management applications or wearable devices. The reception unit performs preprocessing (e.g., missing value imputation, unit unification, outlier detection) on these data and inputs them into an AI model (e.g., MLP for feature extraction of diet, exercise, and sleep, plus classification model for filtering judgment). Examples of AI input include: (1) diet: high fat, low vegetables; exercise: walking once a week; sleep: average 6 hours; (2) diet: well-balanced; exercise: running every day; sleep: average 7.5 hours; (3) diet: high carbohydrate, frequent eating out; exercise: almost none; sleep: irregular, etc. The AI model generates outputs such as prioritized input item list (e.g., blood glucose, cholesterol, body weight as health examination items), input recommendation score (0-1), and input order (e.g., priority, normal, deferred) based on these inputs. Output examples include: (1) priority items: blood glucose, cholesterol; recommendation score: 0.90; order: priority; (2) priority items: cardiopulmonary function, muscle strength; recommendation score: 0.80; order: normal; (3) priority items: body weight, blood pressure; recommendation score: 0.85; order: priority, etc. These outputs are used for automatic rearrangement of input items on the reception screen, highlighting of required input items, and generation of input support messages. Internal processing of the AI model includes feature extraction of each lifestyle habit data, calculation of relevance scores with health examination items, hybrid judgment of rule-based and machine learning, and learning by loss functions (e.g., cross-entropy loss). When not using AI, similar processing is performed using simple threshold judgment or rule-based item selection for diet, exercise, and sleep. Technical effects include that by utilizing AI, the reception unit can analyze the user's lifestyle habits and dietary content in high-dimensional space and realize personalization, efficiency, reduction of input burden, and improvement of input accuracy for health examination input items. Application fields include health examination data reception systems, patient input support for medical institutions, corporate health management platforms, and municipal health management services.

The reception unit can estimate the user's emotions and determine the priority of health examination results to be input based on the estimated emotions of the user. The reception unit may, for example, estimate the user's emotions and determine the input priority of health examination results based on the estimated emotions. Emotion estimation may be performed, for example, using facial recognition, voice analysis, or questionnaire results. Facial recognition may be performed, for example, by analyzing the user's facial expressions captured by a camera. Voice analysis may be performed, for example, by analyzing the tone and speed of the user's voice. Questionnaire results may be estimated, for example, based on the user's answers to questions about emotions. Thus, the reception unit can determine the input priority of health examination results according to the user's emotions. For example, if the user feels stressed, important items are input preferentially. If the user is relaxed, detailed items are input. Furthermore, if the user is in a hurry, only the most important items are input. Emotion estimation may be realized, for example, using an emotion engine or generative AI with emotion estimation functions. Generative AI may include, for example, text generation AI (e.g., LLM) or multimodal generative AI, but is not limited to these examples. Some or all of the above-described processing in the reception unit may be performed using AI or may be performed without using AI. For example, the reception unit may input the user's emotion data into AI and have the AI execute the determination of input priority. Specifically, the reception unit acquires multiple modality data for emotion estimation of the user. For facial recognition, camera images (RGB image tensor, resolution 128×128 pixels, 3 channels); for voice analysis, voice waveform data (1D time series array, sampling rate 16 kHz, length 3-10 seconds); for questionnaire results, text data (free description or selection format, with category labels) are acquired. The reception unit applies preprocessing such as face region extraction, voice noise removal, and text normalization to these data and inputs them into a multimodal AI model (e.g., integrated model of CNN for images, RNN for voice, and Transformer for text). Examples of AI input include: (1) camera image: smiling face, voice: calm tone, questionnaire: “I am relaxed now”; (2) camera image: frowning face, voice: fast and high-pitched, questionnaire: “I am a little irritated”; (3) camera image: expressionless, voice: monotone, questionnaire: “Busy,” etc. The AI model integrates these inputs and generates outputs such as emotion label (e.g., relaxed, stressed, busy), emotion score (continuous value from 0 to 1), and input priority list (e.g., important items, detailed items, minimal items). Output examples include: (1) emotion: relaxed, score: 0.85, priority list: detailed items; (2) emotion: stressed, score: 0.70, priority list: important items; (3) emotion: busy, score: 0.90, priority list: minimal items, etc. These outputs are used in subsequent processing of the reception unit, such as rearrangement of items on the input screen, highlighting of required input items, and generation of input support messages. Internal processing of the AI model includes feature extraction layers for each modality, concatenation of multidimensional feature vectors in the integration layer, and implementation of multitask learning for emotion classification, score regression, and priority recommendation in the fully connected layer. Training uses actual user input history and emotion-labeled datasets, and weights are optimized using cross-entropy loss and mean squared error as loss functions. When not using AI, similar estimation is performed using threshold judgment of facial feature points, rule-based judgment of voice pitch and speed, and simple matching of questionnaire responses. Technical effects include that by utilizing AI, the reception unit can estimate the user's emotional state with high accuracy and in real time, and automatically adjust the optimal input priority, thereby reducing input stress, improving input completion rate, optimizing user experience, and enhancing overall system efficiency. Application fields include health examination data reception systems, patient input support for medical institutions, corporate health management platforms, and municipal health management services.

The reception unit can prioritize the input of highly relevant data based on the user's geographic location information when inputting health examination results. The reception unit may, for example, prioritize the input of highly relevant data based on the user's geographic location information. Geographic location information may be obtained, for example, using GPS data or location information services. GPS data may be obtained, for example, from the user's smartphone or wearable device. Location information services may be obtained, for example, using location information services provided via the Internet. Thus, the reception unit can prioritize the input of highly relevant data based on the user's geographic location information. For example, if the user lives in a high-altitude area, data on oxygen saturation is prioritized. If the user lives in an urban area, data related to air pollution is prioritized. Furthermore, if the user lives by the sea, data related to salt intake is prioritized. Some or all of the above-described processing in the reception unit may be performed using AI or may be performed without using AI. For example, the reception unit may input the user's geographic location information into AI and have the AI execute the prioritization of highly relevant data input. Specifically, the reception unit automatically acquires the user's geographic location information (e.g., numerical vectors of latitude and longitude, elevation information, category labels such as urban, rural, coastal) from smartphones or wearable devices. The reception unit applies geographic feature extraction (e.g., normalization of elevation, urban classification, climate zone assignment) and linkage with external environmental databases (e.g., air pollution index, salt intake risk map) to these data for preprocessing, and inputs them into an AI model (e.g., MLP for geographic information feature extraction plus health examination item priority estimation model). Examples of AI input include: (1) latitude: −13.2, longitude: −72.5, elevation: 2500 m, category: high-altitude; (2) latitude: 35.7, longitude: 139.7, elevation: 40 m, category: urban; (3) latitude: 34.7, longitude: 135.3, elevation: 5 m, category: coastal, etc. The AI model generates outputs such as prioritized input item list (e.g., oxygen saturation, blood pressure, cardiopulmonary function as health examination items), input recommendation score (0-1), and input order (e.g., priority, normal, deferred) based on these inputs. Output examples include: (1) priority items: oxygen saturation, blood pressure; recommendation score: 0.95; order: priority; (2) priority items: air pollution-related items, respiratory function; recommendation score: 0.90; order: priority; (3) priority items: salt intake, renal function; recommendation score: 0.85; order: priority, etc. These outputs are used for automatic rearrangement of input items on the reception screen, highlighting of required input items, and generation of input support messages. Internal processing of the AI model includes extraction of geographic features, calculation of relevance scores with health examination items, hybrid judgment of rule-based and machine learning, and learning by loss functions (e.g., cross-entropy loss). When not using AI, similar processing is performed using simple rule-based item selection or threshold judgment based on geographic conditions. Technical effects include that by utilizing AI, the reception unit can analyze the user's geographic location information in high-dimensional space and realize personalization, efficiency, reduction of input burden, and improvement of input accuracy for health examination input items. Application fields include health examination data reception systems, region-specific input support for medical institutions, corporate health management platforms, and municipal health management services.

The reception unit can analyze the user's social media activities and input relevant data when inputting health examination results. The reception unit may, for example, analyze the user's social media activities and input relevant data. Information on social media activities may be obtained, for example, from post content, number of likes, and number of followers. Post content may be analyzed, for example, by analyzing the content posted by the user on social media. The number of likes may be counted, for example, by counting the number of likes on the user's posts. The number of followers may be counted, for example, by counting the number of the user's followers. Thus, the reception unit can input relevant data based on the user's social media activities. For example, if the user posts about exercise on social media, data related to exercise is prioritized. If the user posts about diet, data related to diet is prioritized. Furthermore, if the user posts about stress, data related to stress is prioritized. Some or all of the above-described processing in the reception unit may be performed using AI or may be performed without using AI. For example, the reception unit may input the user's social media activity data into AI and have the AI execute the input of relevant data. Specifically, the reception unit automatically acquires the user's social media activity data (e.g., post text, images, videos, number of likes, number of followers as structured and unstructured data) via APIs, etc. The reception unit applies preprocessing such as text analysis by natural language processing (e.g., topic extraction, sentiment analysis), image recognition (e.g., classification of exercise, diet, and stress-related images), and normalization of numerical data (e.g., scaling of number of likes and followers) to these data and inputs them into an AI model (e.g., Transformer for text, CNN for images, and MLP for integration of numerical features). Examples of AI input include: (1) post: “Ran 10 km today,” image: running shoes, number of likes: 50; (2) post: “Eating out a lot recently,” image: restaurant food, number of likes: 30; (3) post: “Work is busy and stressful,” image: desk work, number of likes: 20, etc. The AI model generates outputs such as relevant health examination item list (e.g., exercise, cardiopulmonary function, diet, blood glucose, stress, blood pressure), input recommendation score (0-1), and input order (e.g., priority, normal, deferred) based on these inputs. Output examples include: (1) priority items: exercise, cardiopulmonary function; recommendation score: 0.90; order: priority; (2) priority items: diet, blood glucose; recommendation score: 0.85; order: priority; (3) priority items: stress, blood pressure; recommendation score: 0.80; order: priority, etc. These outputs are used for automatic rearrangement of input items on the reception screen, highlighting of required input items, and generation of input support messages. Internal processing of the AI model includes multimodal feature extraction of text, images, and numerical data, calculation of relevance scores with health examination items, and learning by loss functions (e.g., cross-entropy loss). When not using AI, similar processing is performed using keyword matching or simple category classification. Technical effects include that by utilizing AI, the reception unit can analyze the user's social media activities in high-dimensional space and realize personalization, efficiency, reduction of input burden, and improvement of input accuracy for health examination input items. Application fields include health examination data reception systems, patient input support for medical institutions, corporate health management platforms, and municipal health management services.

The analysis unit can estimate the user's emotions and adjust the method of presenting analysis based on the estimated emotions of the user. The analysis unit may, for example, estimate the user's emotions and adjust the method of presenting analysis based on the estimated emotions. Emotion estimation may be performed, for example, using facial recognition, voice analysis, or questionnaire results. Facial recognition may be performed, for example, by analyzing the user's facial expressions captured by a camera. Voice analysis may be performed, for example, by analyzing the tone and speed of the user's voice. Questionnaire results may be estimated, for example, based on the user's answers to questions about emotions. Thus, the analysis unit can adjust the method of presenting analysis according to the user's emotions. For example, if the user is relaxed, detailed analysis results are provided. If the user feels stressed, concise analysis results are provided. Furthermore, if the user is excited, visually appealing analysis results are provided. Emotion estimation may be realized, for example, using an emotion engine or generative AI with emotion estimation functions. Generative AI may include, for example, text generation AI (e.g., LLM) or multimodal generative AI, but is not limited to these examples. Some or all of the above-described processing in the analysis unit may be performed using AI or may be performed without using AI. For example, the analysis unit may input the user's emotion data into AI and have the AI execute the adjustment of the method of presenting analysis. Specifically, the analysis unit acquires multiple modality data for emotion estimation of the user. For facial recognition, camera images (RGB image tensor, resolution 128×128 pixels, 3 channels); for voice analysis, voice waveform data (1D time series array, sampling rate 16 kHz, length 3-10 seconds); for questionnaire results, text data (free description or selection format, with category labels) are acquired. The analysis unit applies preprocessing such as face region extraction, voice noise removal, and text normalization to these data and inputs them into a multimodal AI model (e.g., integrated model of CNN for images, RNN for voice, and Transformer for text). Examples of AI input include: (1) camera image: smiling face, voice: calm tone, questionnaire: “I am relaxed now”; (2) camera image: frowning face, voice: fast and high-pitched, questionnaire: “I am a little irritated”; (3) camera image: expressionless, voice: monotone, questionnaire: “Busy,” etc. The AI model integrates these inputs and generates outputs such as emotion label (e.g., relaxed, stressed, excited), emotion score (continuous value from 0 to 1), and recommended presentation method (e.g., detailed, concise, visually emphasized). Output examples include: (1) emotion: relaxed, score: 0.85, recommended presentation: detailed; (2) emotion: stressed, score: 0.70, recommended presentation: concise; (3) emotion: excited, score: 0.90, recommended presentation: visually emphasized, etc. These outputs are input to subsequent processing modules of the analysis unit, such as text generation modules or graph generation modules, and automatically generate presentation methods optimized for the user's emotional state (e.g., detailed numerical explanations, summary of key points only, infographics or color-enhanced graphs). Internal processing of the AI model includes feature extraction layers for each modality, concatenation of multidimensional feature vectors in the integration layer, and implementation of multitask learning for emotion classification, score regression, and presentation method recommendation in the fully connected layer. Training uses actual user input history, emotion-labeled datasets, and presentation method preference data, and weights are optimized using cross-entropy loss and mean squared error as loss functions. When not using AI, similar estimation is performed using threshold judgment of facial feature points, rule-based judgment of voice pitch and speed, and simple matching of questionnaire responses, and the presentation method is switched by predefined rules. Technical effects include that by utilizing AI, the analysis unit can estimate the user's emotional state with high accuracy and in real time, and automatically optimize the method of presenting analysis results, thereby improving user understanding, satisfaction, behavioral change rate, reducing stress, and optimizing overall user experience of the system. Application fields include health examination result analysis systems, patient explanation support for medical institutions, corporate health management platforms, and municipal health management services.

The analysis unit can adjust the level of detail of analysis based on the importance of the health examination results during analysis. The analysis unit may, for example, adjust the level of detail of analysis based on the importance of the health examination results. Evaluation of importance may be performed, for example, based on physician evaluation or comparison with past data. Physician evaluation may be performed, for example, based on physician comments or evaluations of health examination results. Comparison with past data may be performed, for example, by comparing the user's past health examination results with current results. Thus, the analysis unit can adjust the level of detail of analysis based on the importance of the health examination results. For example, detailed analysis is performed for important health examination results. For less important health examination results, concise analysis is performed. Furthermore, the level of detail of analysis is adjusted stepwise according to the importance of the health examination results. Some or all of the above-described processing in the analysis unit may be performed using AI or may be performed without using AI. For example, the analysis unit may input the importance of health examination results into AI and have the AI execute the adjustment of the level of detail of analysis. Specifically, the analysis unit integrates health examination result data (e.g., blood pressure, heart rate, body weight, height, blood test values, etc., as numerical vectors, each element being a real value, with dimensionality of about 5 to 20), physician evaluation data (e.g., importance label, comment text), and comparison results with past data (e.g., change amount, abnormality score) as input. The analysis unit applies preprocessing (e.g., normalization, outlier detection, vectorization of text) to these data and inputs them into an AI model (e.g., MLP for importance estimation plus generation model for detail control). Examples of AI input include: (1) blood pressure: 150/95, physician evaluation: important, compared to past: +20; (2) blood glucose: 90 mg/dL, physician evaluation: normal, compared to past: ±0; (3) cholesterol: 250 mg/dL, physician evaluation: important, compared to past: +30, etc. The AI model generates outputs such as analysis detail label (e.g., detailed, normal, concise), analysis content generation parameters (e.g., length of explanation, type of graph, presence or absence of annotation) based on these inputs. Output examples include: (1) detail: detailed, explanation: 500 characters, graph: line+bar graph, annotation: present; (2) detail: normal, explanation: 200 characters, graph: bar graph, annotation: absent; (3) detail: concise, explanation: 50 characters, graph: none, annotation: absent, etc. These outputs are input to the analysis result generation module, and the granularity and presentation method of analysis content presented to the user are automatically adjusted. Internal processing of the AI model includes weighting of importance features, threshold judgment of change amount compared to past data, semantic extraction of physician comments by natural language processing, optimization of detail control parameters, and learning by loss functions (e.g., cross-entropy loss, mean squared error). When not using AI, the level of detail is switched by predefined rules or threshold judgment. Technical effects include that by utilizing AI, the analysis unit can analyze the importance of health examination results in high-dimensional space and automatically optimize the granularity of analysis content, thereby improving user understanding, emphasizing important information, reducing unnecessary information, and enhancing overall system efficiency. Application fields include health examination result analysis systems, patient explanation support for medical institutions, corporate health management platforms, and municipal health management services.

The analysis unit can apply different analysis algorithms according to the category of health examination results during analysis. The analysis unit may, for example, apply different analysis algorithms according to the category of health examination results. Classification of categories may be performed, for example, based on blood tests, electrocardiograms, or physical fitness tests. The blood test category may include, for example, blood glucose, cholesterol, liver function tests, etc. The electrocardiogram category may include, for example, heart rate, analysis of electrocardiogram waveforms, etc. The physical fitness test category may include, for example, cardiopulmonary function tests and muscle strength tests. Thus, the analysis unit can apply different analysis algorithms according to the category of health examination results. For example, cardiovascular health examination results are analyzed using algorithms specialized for the cardiovascular system. Digestive system health examination results are analyzed using algorithms specialized for the digestive system. Furthermore, respiratory system health examination results are analyzed using algorithms specialized for the respiratory system. Some or all of the above-described processing in the analysis unit may be performed using AI or may be performed without using AI. For example, the analysis unit may input the category of health examination results into AI and have the AI execute the application of different analysis algorithms. Specifically, the analysis unit classifies health examination result data (e.g., blood test values, electrocardiogram waveform data, physical fitness test scores as various data types) by category and selectively applies AI models or algorithms optimized for each category (e.g., MLP for blood tests, 1D CNN for electrocardiograms, regression model for physical fitness tests). Examples of AI input include: (1) blood test: blood glucose 110 mg/dL, cholesterol 180 mg/dL; (2) electrocardiogram waveform: 500 samples of time series data; (3) physical fitness test: cardiopulmonary function score 80, muscle strength score 70, etc. The AI model automatically applies different algorithms according to the input category, such as anomaly detection and risk assessment by multilayer perceptron for blood test data, waveform pattern classification and arrhythmia detection by 1D convolutional neural network for electrocardiogram data, and physical age estimation and exercise ability evaluation by regression model for physical fitness test data, and outputs category-specific analysis results (e.g., risk score, anomaly label, recommended exercise intensity). Output examples include: (1) blood test: risk score 0.2, no anomaly; (2) electrocardiogram: anomaly label “arrhythmia”; (3) physical fitness test: estimated physical age 35 years, recommended exercise intensity “moderate,” etc. These outputs are summarized as comprehensive health analysis results for the user in subsequent integration modules. Internal processing of the AI model includes category determination logic, preprocessing for each data type (e.g., waveform normalization, feature extraction), algorithm selection mechanism, and learning by loss functions (e.g., cross-entropy loss, mean squared error). When not using AI, predefined rule-based processing or statistical methods are applied for each category. Technical effects include that by utilizing AI, the analysis unit can automatically select and apply optimal analysis algorithms for diverse categories of health examination results, greatly improving analysis accuracy, speed, and flexibility. Application fields include health examination result analysis systems, automatic diagnosis support for medical institutions, corporate health management platforms, and municipal health management services.

The analysis unit can estimate the user's emotions and adjust the length of analysis based on the estimated emotions of the user. The analysis unit may, for example, estimate the user's emotions and adjust the length of analysis based on the estimated emotions. Emotion estimation may be performed, for example, using facial recognition, voice analysis, or questionnaire results. Facial recognition may be performed, for example, by analyzing the user's facial expressions captured by a camera. Voice analysis may be performed, for example, by analyzing the tone and speed of the user's voice. Questionnaire results may be estimated, for example, based on the user's answers to questions about emotions. Thus, the analysis unit can adjust the length of analysis according to the user's emotions. For example, if the user is in a hurry, a short and concise analysis is provided. If the user is relaxed, a detailed analysis is provided. Furthermore, if the user is excited, a visually appealing analysis is provided. Emotion estimation may be realized, for example, using an emotion engine or generative AI with emotion estimation functions. Generative AI may include, for example, text generation AI (e.g., LLM) or multimodal generative AI, but is not limited to these examples. Some or all of the above-described processing in the analysis unit may be performed using AI or may be performed without using AI. For example, the analysis unit may input the user's emotion data into AI and have the AI execute the adjustment of the length of analysis. Specifically, the analysis unit acquires camera images (RGB image tensor, resolution 128×128 pixels, 3 channels), voice waveform data (1D time series array, sampling rate 16 kHz, length 3-10 seconds), and questionnaire text (with category labels) for emotion estimation, and after preprocessing (face region extraction, voice noise removal, text normalization), inputs them into a multimodal AI model (integrated model of CNN for images, RNN for voice, and Transformer for text). Examples of AI input include: (1) camera image: smiling face, voice: calm tone, questionnaire: “I am relaxed now”; (2) camera image: frowning face, voice: fast and high-pitched, questionnaire: “In a hurry”; (3) camera image: wide-open eyes, voice: excited, questionnaire: “Excited,” etc. The AI model integrates these inputs and generates outputs such as emotion label (relaxed, stressed, excited, in a hurry), emotion score (0-1), and recommended analysis length (e.g., short, medium, long). Output examples include: (1) emotion: relaxed, score: 0.85, analysis length: long; (2) emotion: in a hurry, score: 0.90, analysis length: short; (3) emotion: excited, score: 0.80, analysis length: medium (visually emphasized), etc. These outputs are input to the analysis result generation module, and the length of analysis content (number of characters in explanation, detail of graphs, presence or absence of annotation) is automatically adjusted. Internal processing of the AI model includes feature extraction for each modality, concatenation of multidimensional feature vectors in the integration layer, multitask learning for emotion classification, score regression, and length recommendation in the fully connected layer, and learning by loss functions (cross-entropy loss, mean squared error). When not using AI, the length is switched by threshold judgment of facial feature points or voice features, or rule-based judgment of questionnaire responses. Technical effects include that by utilizing AI, the analysis unit can estimate the user's emotional state with high accuracy and in real time, and automatically optimize the length of analysis content, thereby improving user understanding, satisfaction, behavioral change rate, reducing stress, and optimizing overall user experience of the system. Application fields include health examination result analysis systems, patient explanation support for medical institutions, corporate health management platforms, and municipal health management services.

The analysis unit can determine the priority of analysis based on the submission timing of health examination results during analysis. The analysis unit may, for example, determine the priority of analysis based on the submission timing of health examination results. Evaluation of submission timing may be performed, for example, based on submission date or submission frequency. Submission date may be evaluated, for example, based on the date the user submitted health examination results. Submission frequency may be evaluated, for example, based on how often the user submits health examination results. Thus, the analysis unit can determine the priority of analysis based on the submission timing of health examination results. For example, recent health examination results are analyzed preferentially. Past health examination results are referenced while emphasizing the latest results. Furthermore, the priority of analysis is adjusted stepwise according to the submission timing of health examination results. Some or all of the above-described processing in the analysis unit may be performed using AI or may be performed without using AI. For example, the analysis unit may input the submission timing of health examination results into AI and have the AI execute the determination of analysis priority. Specifically, the analysis unit acquires health examination result data (e.g., blood pressure, heart rate, body weight, height, blood test values, etc., as numerical vectors, each element being a real value, with dimensionality of about 5 to 20), submission date (timestamp), and submission frequency (number of submissions in the past year, etc.) from the database. The analysis unit applies preprocessing (e.g., normalization of dates, calculation of frequency score) to these data and inputs them into an AI model (e.g., MLP for submission timing priority estimation plus time series feature extraction model). Examples of AI input include: (1) blood pressure: 120/80, submission date: 2024 Jun. 1, frequency: once a year; (2) blood glucose: 95 mg/dL, submission date: 2024 May 15, frequency: twice a year; (3) cholesterol: 210 mg/dL, submission date: 2023 Dec. 1, frequency: once a year, etc. The AI model generates outputs such as analysis priority score (0-1), priority label (e.g., high, medium, low), and analysis order (e.g., first, second, third) based on these inputs. Output examples include: (1) priority: high, order: 1; (2) priority: medium, order: 2; (3) priority: low, order: 3, etc. These outputs are input to the analysis queue management module, and the overall analysis processing order of the system is automatically optimized. Internal processing of the AI model includes weighting of submission date and frequency, extraction of time series features, calculation of priority score, and learning by loss functions (cross-entropy loss, mean squared error). When not using AI, priority is determined by simple rule-based judgment based on recency of submission date and frequency. Technical effects include that by utilizing AI, the analysis unit can analyze submission timing and frequency of health examination results in high-dimensional space and automatically optimize the priority of analysis processing, thereby quickly reflecting the latest data, improving user satisfaction, and enhancing overall system efficiency. Application fields include health examination result analysis systems, automatic diagnosis support for medical institutions, corporate health management platforms, and municipal health management services.

The analysis unit can adjust the order of analysis based on the relevance of health examination results during analysis. The analysis unit may, for example, adjust the order of analysis based on the relevance of health examination results. Evaluation of relevance may be performed, for example, based on results in the same category or relevance to past data. Results in the same category may be evaluated, for example, based on results belonging to the same category, such as blood test results or electrocardiogram results. Relevance to past data may be evaluated, for example, by comparing the user's past health examination results with current results. Thus, the analysis unit can adjust the order of analysis based on the relevance of health examination results. For example, health examination results with high relevance are analyzed preferentially. Health examination results with low relevance are deferred. Furthermore, the order of analysis is adjusted stepwise according to the relevance of health examination results. Some or all of the above-described processing in the analysis unit may be performed using AI or may be performed without using AI. For example, the analysis unit may input the relevance of health examination results into AI and have the AI execute the adjustment of analysis order. Specifically, the analysis unit inputs health examination result data (e.g., blood test values, electrocardiogram waveforms, physical fitness test scores as various data types), category labels (e.g., blood, electrocardiogram, physical fitness), and relevance scores to past data (e.g., cosine similarity, change score). The analysis unit applies preprocessing (e.g., normalization by category, calculation of relevance) to these data and inputs them into an AI model (e.g., MLP for relevance estimation plus order optimization algorithm). Examples of AI input include: (1) blood test: blood glucose 110 mg/dL, category: blood, compared to past: +10; (2) electrocardiogram: abnormal waveform, category: electrocardiogram, compared to past: no change; (3) physical fitness test: score 70, category: physical fitness, compared to past: −5, etc. The AI model generates outputs such as analysis order label (e.g., priority, normal, deferred), order index (e.g., 1, 2, 3), and relevance score (0-1) based on these inputs. Output examples include: (1) order: 1, relevance: 0.95; (2) order: 2, relevance: 0.80; (3) order: 3, relevance: 0.60, etc. These outputs are input to the analysis queue management module, and the overall analysis processing order of the system is automatically optimized. Internal processing of the AI model includes calculation of relevance between categories, scoring of similarity to past data, order optimization algorithm (e.g., reinforcement learning-based order determination), and learning by loss functions (cross-entropy loss, mean squared error). When not using AI, order is determined by simple rule-based judgment based on category or past data. Technical effects include that by utilizing AI, the analysis unit can analyze the relevance of health examination results in high-dimensional space and automatically optimize the order of analysis processing, thereby quickly extracting important information, improving user satisfaction, and enhancing overall system efficiency. Application fields include health examination result analysis systems, automatic diagnosis support for medical institutions, corporate health management platforms, and municipal health management services.

The proposal unit can estimate the user's emotions and adjust the method of presenting proposals based on the estimated emotions of the user. The proposal unit may, for example, estimate the user's emotions and adjust the method of presenting proposals based on the estimated emotions. Emotion estimation may be performed, for example, using facial recognition, voice analysis, or questionnaire results. Facial recognition may be performed, for example, by analyzing the user's facial expressions captured by a camera. Voice analysis may be performed, for example, by analyzing the tone and speed of the user's voice. Questionnaire results may be estimated, for example, based on the user's answers to questions about emotions. Thus, the proposal unit can adjust the method of presenting proposals according to the user's emotions. For example, if the user is relaxed, detailed proposals are provided. If the user feels stressed, concise proposals are provided. Furthermore, if the user is excited, visually appealing proposals are provided. Emotion estimation may be realized, for example, using an emotion engine or generative AI with emotion estimation functions. Generative AI may include, for example, text generation AI (e.g., LLM) or multimodal generative AI, but is not limited to these examples. Some or all of the above-described processing in the proposal unit may be performed using AI or may be performed without using AI. For example, the proposal unit may input the user's emotion data into AI and have the AI execute the adjustment of the method of presenting proposals. Specifically, the proposal unit acquires multiple modality data for emotion estimation of the user. For facial recognition, camera images (RGB image tensor, resolution 128×128 pixels, 3 channels); for voice analysis, voice waveform data (1D time series array, sampling rate 16 kHz, length 3-10 seconds); for questionnaire results, text data (free description or selection format, with category labels) are acquired. The proposal unit applies preprocessing such as face region extraction, voice noise removal, and text normalization to these data and inputs them into a multimodal AI model (e.g., integrated model of CNN for images, RNN for voice, and Transformer for text). Examples of AI input include: (1) camera image: smiling face, voice: calm tone, questionnaire: “I am relaxed now”; (2) camera image: frowning face, voice: fast and high-pitched, questionnaire: “I am a little irritated”; (3) camera image: expressionless, voice: monotone, questionnaire: “Busy,” etc. The AI model integrates these inputs and generates outputs such as emotion label (e.g., relaxed, stressed, excited), emotion score (continuous value from 0 to 1), and recommended presentation method (e.g., detailed, concise, visually emphasized). Output examples include: (1) emotion: relaxed, score: 0.85, recommended presentation: detailed; (2) emotion: stressed, score: 0.70, recommended presentation: concise; (3) emotion: excited, score: 0.90, recommended presentation: visually emphasized, etc. These outputs are input to subsequent processing modules of the proposal unit, such as proposal content generation modules or graph generation modules, and automatically generate presentation methods optimized for the user's emotional state (e.g., detailed explanation, summary of key points only, infographics or color-enhanced graphs). Internal processing of the AI model includes feature extraction layers for each modality, concatenation of multidimensional feature vectors in the integration layer, and implementation of multitask learning for emotion classification, score regression, and presentation method recommendation in the fully connected layer. Training uses actual user input history, emotion-labeled datasets, and presentation method preference data, and weights are optimized using cross-entropy loss and mean squared error as loss functions. When not using AI, similar estimation is performed using threshold judgment of facial feature points, rule-based judgment of voice pitch and speed, and simple matching of questionnaire responses, and the presentation method is switched by predefined rules. Technical effects include that by utilizing AI, the proposal unit can estimate the user's emotional state with high accuracy and in real time, and automatically optimize the method of presenting proposal content, thereby improving user understanding, satisfaction, behavioral change rate, reducing stress, and optimizing overall user experience of the system. Application fields include challenge proposal systems based on health examination results, patient explanation support for medical institutions, corporate health management platforms, and municipal health management services.

The proposal unit can adjust the level of detail of proposals at the time of proposal based on the importance of the challenge. For example, the proposal unit adjusts the level of detail of proposals according to the importance of the challenge. The evaluation of importance is performed, for example, based on the impact on health and the difficulty of achievement. The impact on health is evaluated, for example, based on the effect the challenge has on the user's health. The difficulty of achievement is evaluated, for example, based on how difficult it is to accomplish the challenge. Thus, the proposal unit can adjust the level of detail of proposals according to the importance of the challenge. For example, detailed proposals are made for important challenges. For challenges that are not so important, concise proposals are made. Furthermore, the level of detail of proposals is gradually adjusted according to the importance of the challenge. Some or all of the above-described processing in the proposal unit may be performed using AI, or may be performed without using AI. For example, the proposal unit can input the importance of the challenge to AI and have the AI execute the adjustment of the level of detail of proposals. Specifically, the proposal unit uses, for importance evaluation of challenges, health examination result data (e.g., numerical vectors such as blood pressure, heart rate, body weight, height, blood test values), challenge content data (e.g., structured data such as exercise type, target distance, reason for recommendation), and health impact scores or achievement difficulty scores (continuous values from 0 to 1, or labels such as low, medium, high) as inputs. The proposal unit normalizes and extracts features from these data, then inputs them into an AI model (e.g., MLP for importance estimation plus a generative model for detail control). Examples of AI inputs include: (1) Challenge: full marathon, health impact: high, difficulty: high; (2) Challenge: 10 km walking, health impact: medium, difficulty: medium; (3) Challenge: stretching, health impact: low, difficulty: low. The AI model generates outputs such as proposal detail labels (e.g., detailed, normal, concise), proposal content generation parameters (e.g., length of explanation, type of graph, presence or absence of annotations), etc. Output examples include: (1) Detail: detailed, explanation: 500 characters, graph: line+bar graph, annotation: present; (2) Detail: normal, explanation: 200 characters, graph: bar graph, annotation: absent; (3) Detail: concise, explanation: 50 characters, graph: none, annotation: none. These outputs are input to the proposal content generation module, and the granularity and expression method of the proposal content presented to the user are automatically adjusted. Internal processing of the AI model includes weighting of importance features, threshold determination for health impact and difficulty, optimization of detail control parameters, and learning using loss functions (cross-entropy loss, mean squared error). If AI is not used, the level of detail is switched by predefined rules or threshold determination. The technical effect is that by utilizing AI, the proposal unit can analyze the importance of challenges in a high-dimensional space and automatically optimize the granularity of proposal content, thereby improving user comprehension, emphasizing important information, reducing unnecessary information, and enhancing overall system efficiency. Application fields include challenge proposal systems based on health examination results, patient explanation support in medical institutions, corporate health management platforms, and municipal health management services.

The proposal unit can apply different proposal algorithms according to the category of the challenge at the time of proposal. For example, the proposal unit applies different proposal algorithms according to the category of the challenge. Categorization is performed, for example, based on exercise, diet, lifestyle habits, etc. The exercise category includes, for example, walking, running, hiking, and so on. The diet category includes, for example, nutritionally balanced meals, specific diet plans, and so on. The lifestyle habits category includes, for example, improvement of sleep patterns, stress management, and so on. Thus, the proposal unit can apply the optimal proposal algorithm according to the category of the challenge. For example, for sports-related challenges, a sports-specific proposal algorithm is applied. For travel-related challenges, a travel-specific proposal algorithm is applied. Furthermore, for health-related challenges, a health-specific proposal algorithm is applied. Some or all of the above-described processing in the proposal unit may be performed using AI, or may be performed without using AI. For example, the proposal unit can input the category of the challenge to AI and have the AI execute the application of different proposal algorithms. Specifically, the proposal unit uses challenge content data (e.g., category labels, exercise type, diet plan name, lifestyle improvement items as structured data), user's health examination results (e.g., numerical vectors), and past challenge history as inputs. The proposal unit classifies these data by category and selectively applies AI models or algorithms optimized for each category (e.g., MLP for exercise proposals, recommender for diet proposals, reinforcement learning model for lifestyle improvement). Examples of AI inputs include: (1) Category: exercise, type: running; (2) Category: diet, plan: low-carb diet; (3) Category: lifestyle, item: sleep improvement. The AI model automatically applies different algorithms according to the input category, such as optimization of exercise intensity, frequency, and target distance for the exercise category; nutrition balance, calorie intake, and recipe recommendation for the diet category; and behavior change plans and reminder generation for the lifestyle category, and outputs category-specific proposal results (e.g., exercise menu, diet plan, lifestyle improvement plan). Output examples include: (1) Exercise: running 30 minutes three times a week; (2) Diet: three low-carb meals per day; (3) Lifestyle: reminder to go to bed at 11 p.m. every night. These outputs are used for proposal screens and notification messages to users. Internal processing of the AI model includes category determination logic, preprocessing for each data type, algorithm selection mechanism, and learning using loss functions (cross-entropy loss, mean squared error). If AI is not used, rule-based processing or statistical methods predefined for each category are applied. The technical effect is that by utilizing AI, the proposal unit can automatically select and apply the optimal proposal algorithm for diverse challenge categories, greatly improving proposal accuracy, speed, and flexibility. Application fields include challenge proposal systems based on health examination results, lifestyle guidance for patients in medical institutions, corporate health management platforms, and municipal health management services.

The proposal unit can estimate the user's emotions and adjust the length of proposals based on the estimated emotions of the user. For example, the proposal unit estimates the user's emotions and adjusts the length of proposals according to the estimated emotions. Emotion estimation is performed, for example, using facial recognition, voice analysis, and questionnaire results. Facial recognition analyzes the user's facial expressions captured by a camera. Voice analysis analyzes the tone and speed of the user's voice. Questionnaire results estimate emotions based on the user's answers to emotion-related questions. Thus, the proposal unit can adjust the length of proposals according to the user's emotions. For example, if the user is in a hurry, a short and concise proposal is made. If the user is relaxed, a detailed proposal is made. Furthermore, if the user is excited, a visually appealing proposal is made. Emotion estimation is realized, for example, using emotion engines or generative AI with emotion estimation functions. Generative AI may include text generation AI (e.g., LLM) or multimodal generative AI, but is not limited to these examples. Some or all of the above-described processing in the proposal unit may be performed using AI, or may be performed without using AI. For example, the proposal unit can input the user's emotion data to AI and have the AI execute the adjustment of proposal length. Specifically, the proposal unit obtains camera images (RGB image tensors, resolution 128×128 pixels, 3 channels), voice waveform data (1D time series array, sampling rate 16 kHz, length 3-10 seconds), and questionnaire text (with category labels) for emotion estimation, and after preprocessing (face region extraction, voice noise removal, text normalization), inputs them into a multimodal AI model (integrated model of CNN for images, RNN for voice, and Transformer for text). Examples of AI inputs include: (1) Camera image: smiling face, voice: calm tone, questionnaire: “I am relaxed”; (2) Camera image: frowning face, voice: fast and high-pitched, questionnaire: “I am in a hurry”; (3) Camera image: wide-open eyes, voice: excited, questionnaire: “I am excited”. The AI model integrates these inputs and generates outputs such as emotion labels (relaxed, stressed, excited, in a hurry), emotion scores (0-1), and recommended proposal length (e.g., short, medium, long). Output examples include: (1) Emotion: relaxed, score: 0.85, proposal length: long; (2) Emotion: in a hurry, score: 0.90, proposal length: short; (3) Emotion: excited, score: 0.80, proposal length: medium (visually emphasized). These outputs are input to the proposal content generation module, and the length of proposal content (number of characters in explanation, level of detail in graphs, presence or absence of annotations, etc.) is automatically adjusted. Internal processing of the AI model includes feature extraction for each modality, concatenation of multidimensional feature vectors in the integration layer, fully connected layers for emotion classification, score regression, and length recommendation in multitask learning, and learning using loss functions (cross-entropy loss, mean squared error). If AI is not used, length is switched by threshold determination of facial feature points, voice features, or rule-based determination of questionnaire answers. The technical effect is that by utilizing AI, the proposal unit can estimate the user's emotional state with high accuracy and in real time, and automatically optimize the length of proposal content, thereby improving user comprehension, satisfaction, behavior change rate, reducing stress, and optimizing the overall user experience of the system. Application fields include challenge proposal systems based on health examination results, patient explanation support in medical institutions, corporate health management platforms, and municipal health management services.

The proposal unit can determine the priority of proposals at the time of proposal based on the submission timing of challenges. For example, the proposal unit determines the priority of proposals according to the submission timing of challenges. Evaluation of submission timing is performed, for example, based on the submission date and submission frequency. The submission date is evaluated, for example, based on the date the user submitted the challenge. Submission frequency is evaluated, for example, based on how often the user submits challenges. Thus, the proposal unit can determine the priority of proposals according to the submission timing of challenges. For example, recent challenges are proposed with priority. Past challenges are referenced while emphasizing the latest challenges. Furthermore, the priority of proposals is gradually adjusted according to the submission timing of challenges. Some or all of the above-described processing in the proposal unit may be performed using AI, or may be performed without using AI. For example, the proposal unit can input the submission timing of challenges to AI and have the AI execute the determination of proposal priority. Specifically, the proposal unit obtains challenge content data (e.g., challenge name, category, relevance to health examination results), submission date (timestamp), and submission frequency (number of submissions in the past year, etc.) from the database. The proposal unit preprocesses these data (date normalization, frequency score calculation) and inputs them into an AI model (e.g., MLP for submission timing priority estimation plus time series feature extraction model). Examples of AI inputs include: (1) Challenge: 10 km marathon, submission date: 2024 Jun. 1, frequency: once a year; (2) Challenge: mountain climbing trip, submission date: 2024 May 15, frequency: twice a year; (3) Challenge: stretching, submission date: 2023 Dec. 1, frequency: once a year. The AI model generates outputs such as proposal priority score (0-1), priority label (e.g., high, medium, low), and proposal order (e.g., first, second, third). Output examples include: (1) Priority: high, order: 1; (2) Priority: medium, order: 2; (3) Priority: low, order: 3. These outputs are input to the proposal queue management module, and the overall proposal processing order of the system is automatically optimized. Internal processing of the AI model includes weighting of submission date and frequency, extraction of time series features, calculation of priority scores, and learning using loss functions (cross-entropy loss, mean squared error). If AI is not used, priority is determined by simple rule-based judgment based on recency of submission date and frequency. The technical effect is that by utilizing AI, the proposal unit can analyze the submission timing and frequency of challenges in a high-dimensional space and automatically optimize the priority of proposal processing, thereby enabling rapid reflection of the latest data, improving user satisfaction, and enhancing overall system efficiency. Application fields include challenge proposal systems based on health examination results, lifestyle guidance for patients in medical institutions, corporate health management platforms, and municipal health management services.

The proposal unit can adjust the order of proposals at the time of proposal based on the relevance of challenges. For example, the proposal unit adjusts the order of proposals according to the relevance of challenges. Evaluation of relevance is performed, for example, based on the relevance to challenges in the same category or to past challenges. Challenges in the same category are evaluated, for example, based on challenges related to exercise or travel that belong to the same category. Relevance to past challenges is evaluated, for example, by comparing the user's past challenges with current challenges. Thus, the proposal unit can adjust the order of proposals according to the relevance of challenges. For example, highly relevant challenges are proposed with priority. Challenges with low relevance are proposed later. Furthermore, the order of proposals is gradually adjusted according to the relevance of challenges. Some or all of the above-described processing in the proposal unit may be performed using AI, or may be performed without using AI. For example, the proposal unit can input the relevance of challenges to AI and have the AI execute the adjustment of proposal order. Specifically, the proposal unit uses challenge content data (e.g., challenge name, category, relevance to health examination results), past challenge history (e.g., list of challenges previously proposed or implemented), and relevance scores (e.g., cosine similarity, category match score, change score) as inputs. The proposal unit preprocesses these data (normalization by category, relevance calculation) and inputs them into an AI model (e.g., MLP for relevance estimation plus order optimization algorithm). Examples of AI inputs include: (1) Challenge: 10 km marathon, category: exercise, change from past: +10; (2) Challenge: mountain climbing trip, category: travel, change from past: none; (3) Challenge: stretching, category: exercise, change from past: −5. The AI model generates outputs such as proposal order label (e.g., priority, normal, postponed), order index (e.g., 1, 2, 3), and relevance score (0-1). Output examples include: (1) Order: 1, relevance: 0.95; (2) Order: 2, relevance: 0.80; (3) Order: 3, relevance: 0.60. These outputs are input to the proposal queue management module, and the overall proposal processing order of the system is automatically optimized. Internal processing of the AI model includes calculation of relevance between categories, similarity scoring with past data, order optimization algorithms (e.g., reinforcement learning-based order determination), and learning using loss functions (cross-entropy loss, mean squared error). If AI is not used, order is determined by simple rule-based judgment based on category or past data. The technical effect is that by utilizing AI, the proposal unit can analyze the relevance of challenges in a high-dimensional space and automatically optimize the proposal processing order, thereby enabling rapid extraction of important information, improving user satisfaction, and enhancing overall system efficiency. Application fields include challenge proposal systems based on health examination results, lifestyle guidance for patients in medical institutions, corporate health management platforms, and municipal health management services.

The system according to the embodiment is not limited to the examples described above and can be variously modified as follows, for example. Specifically, the system can allow diverse variations in the functions and data flow of each component such as the reception unit, analysis unit, and proposal unit, the architecture of AI models, types of input data, output formats, user interfaces, linkage with external databases, connection methods with sensor devices, etc., according to implementation requirements and operating environments. For example, the system may be configured so that the reception unit receives not only health examination results but also real-time biometric data from wearable devices (e.g., heart rate variability, step count, calories burned, etc.) and lifestyle records from smartphone apps (e.g., self-reported data on diet, exercise, sleep, stress, etc.). The analysis unit may be configured to combine not only a single AI model (e.g., multilayer perceptron) but also multiple AI models (e.g., 1D CNN for ECG analysis, Transformer for diet record analysis, LSTM for time series prediction) to perform optimal analysis for each data type. Furthermore, the proposal unit can generate personalized challenges and health advice by integrally considering not only the user's health condition and interests/concerns but also geographic location information, weather information, social network information, feedback from medical institutions, etc. Various learning methods for AI models, such as supervised learning, reinforcement learning, transfer learning, and self-supervised learning, can be applied, and loss functions and optimization algorithms can be selected according to the application. Database configurations can be flexibly designed according to security requirements and operating costs, such as cloud-based distributed databases, on-premises databases, and local storage on edge devices. User interfaces can adopt various forms such as web browsers, smartphone apps, voice dialogue interfaces, and chatbots. Due to these diverse possibilities for modification, the system can realize optimal configurations in a wide range of application fields without depending on specific implementations or operating environments, such as medical institutions, companies, municipalities, sports clubs, and personal health management services. The technical effect is that the flexibility, scalability, operational efficiency, and user adaptability of the system are greatly improved, and future technological evolution and adaptation to new data sources become easier. As a result, a causal relationship is established in which the accuracy, speed, and user experience of the entire process of collecting, analyzing, and proposing health examination data are continuously improved. Application fields include health examination result management systems, patient support platforms for medical institutions, corporate health management support, municipal health policy promotion, sports club member management, and personal health promotion applications.

The reception unit can refer to the user's past exercise history and dietary history when receiving the user's health examination results, thereby improving the accuracy of input data. For example, abnormal values can be detected by comparing current health examination results with exercise data recorded by the user in the past. The user's dietary history can also be referenced to point out imbalances in nutrition. Furthermore, an automatic completion function can be provided to reduce input effort based on data previously entered by the user. Thus, the reception unit can utilize the user's past data to achieve more accurate and efficient data input. Specifically, the reception unit obtains, for each user, exercise history data (e.g., time series vectors of date, exercise type, exercise intensity, duration, calories burned, etc., with history length of 1 to 365 days), dietary history data (e.g., table data including food names, quantities, and nutrient information for three meals per day, for one week to one year), and past health examination input data (e.g., history vectors of blood pressure, body weight, blood test values, etc.) from the database. The reception unit performs preprocessing such as time series feature extraction (e.g., moving average, trend score, anomaly detection), nutrition balance evaluation (e.g., calculation of major nutrient ratios, comparison with recommended intake), and input pattern analysis (e.g., aggregation of frequent values and input times) on these data, and inputs them into an AI model (e.g., time series RNN plus AutoEncoder for anomaly detection plus MLP for nutrition balance judgment). Examples of AI inputs include: (1) Exercise history for the past 30 days plus latest health examination data; (2) Dietary history for the past week plus blood test values; (3) Last five health examination input values plus exercise and dietary history. The AI model generates outputs such as anomaly detection labels (e.g., normal, caution, abnormal), nutrition balance bias indication (e.g., category labels such as excess fat, lack of vegetables), and automatic completion candidate values (e.g., estimated body weight, blood pressure). Output examples include: (1) Anomaly: weight increase of 10 kg, caution; (2) Nutrition balance: excess fat; (3) Completion candidate: blood pressure 120/80. These outputs are used for input assistance on the reception screen, anomaly warning display, automatic completion of input items, and sending reminder notifications. Internal processing of the AI model includes feature extraction from time series data using LSTM or GRU, anomaly score calculation using AutoEncoder, category classification using MLP for nutrition balance judgment, integration by fully connected layers, and learning using loss functions (e.g., cross-entropy loss, mean squared error). If AI is not used, similar processing is performed by simple mean value completion of past data, rule-based judgment of nutrient ratios, and threshold determination of abnormal values. The technical effect is that by utilizing AI, the reception unit can analyze each user's exercise, dietary, and health examination history in a high-dimensional space, and achieve improved accuracy of input data, automation of anomaly detection, efficiency of input operations, reduction of input errors, and enhancement of user experience. Application fields include health examination data reception systems, patient input support in medical institutions, corporate health management platforms, and municipal health management services.

The analysis unit can take into account the user's genetic information when analyzing the user's health examination results. For example, the risk of specific diseases can be evaluated based on family history and genetic risk. In addition, optimal exercise plans and diet plans can be proposed to the user based on genetic information. Furthermore, genetic information can be used to predict long-term changes in the user's health condition and provide preventive advice. Thus, the analysis unit can utilize the user's genetic information to achieve more personalized health management. Specifically, the analysis unit integrates and inputs, for each user, genetic information data (e.g., SNP sequence data, presence or absence of specific gene polymorphisms, family history labels, genetic risk scores as vectors), health examination result data (e.g., numerical vectors such as blood pressure, blood glucose, cholesterol, etc.), and past health examination history. The analysis unit preprocesses these data (e.g., binarization of gene polymorphisms, normalization of risk scores, categorization of family history, standardization of health examination values), and inputs them into an AI model (e.g., genetic information integrated MLP plus classification model for disease risk prediction plus time series RNN for long-term prediction). Examples of AI inputs include: (1) SNP sequence: rs1234-TT, family history: diabetes present, health examination value: blood glucose 110 mg/dL; (2) Genetic risk score: high, family history: no heart disease, health examination value: blood pressure 120/80; (3) Gene polymorphism: APOE-ε4, family history: Alzheimer's present, health examination value: cholesterol 250 mg/dL. The AI model generates outputs such as disease risk evaluation (e.g., high, medium, low risk of diabetes), optimal exercise plan (e.g., recommendation for aerobic exercise), optimal diet plan (e.g., recommendation for low fat, high dietary fiber), long-term health condition prediction (e.g., estimated blood glucose value in five years), and preventive advice (e.g., recommendation for regular checkups). Output examples include: (1) Risk: high, exercise: walking three times a week, diet: vegetable-centered; (2) Risk: medium, exercise: strength training, diet: balanced; (3) Risk: low, exercise: stretching, diet: free. These outputs are transferred to subsequent proposal units and user health management screens, and used as basic data for personalized health advice and challenge proposals. Internal processing of the AI model includes integration of features from genetic information and health examination values, disease risk classification, time series prediction using RNN for long-term prediction, and learning using loss functions (cross-entropy loss, mean squared error). If AI is not used, similar processing is performed by rule-based judgment of genetic risk or simple category branching of family history. The technical effect is that by utilizing AI, the analysis unit can integrally analyze each user's genetic background and health examination data in a high-dimensional space, enabling disease risk evaluation, generation of individually optimized plans, improvement of long-term prediction accuracy, and realization of preventive medicine. Application fields include health examination result analysis systems, health management services utilizing genetic information, personalized diagnosis support in medical institutions, corporate health management platforms, and municipal health policy promotion.

The proposal unit can take into account the user's living environment when making proposals based on the user's health examination results. For example, if the user lives in an urban area, an exercise plan suitable for the urban environment is proposed. If the user lives in a rural area, an exercise plan utilizing the natural environment can also be proposed. Furthermore, appropriate diet plans and stress management methods can be proposed based on the user's living environment. Thus, the proposal unit can make more realistic and feasible proposals by considering the user's living environment. Specifically, the proposal unit integrates and inputs the user's geographic location information (e.g., latitude, longitude, altitude, category labels such as urban/rural/coastal), surrounding environment data (e.g., temperature, humidity, air pollution index, presence of parks/gyms/natural environments), housing type (e.g., apartment, detached house, share house), and health examination result data (e.g., physical fitness score, health risk label). The proposal unit preprocesses these data (e.g., normalization of geographic features, scaling of environmental data, one-hot encoding of housing categories), and inputs them into an AI model (e.g., geographic information integrated MLP plus environment-adaptive proposal generation model). Examples of AI inputs include: (1) Urban area, high-rise apartment, 10-minute walk to park, physical fitness score 70; (2) Rural area, detached house, abundant natural environment, physical fitness score 85; (3) Coastal area, share house, no gym, physical fitness score 60. The AI model generates outputs such as optimal exercise plan (e.g., urban area: indoor training plus park walking; rural area: hiking plus farm work; coastal area: beach running), optimal diet plan (e.g., urban area: focus on balanced eating out; rural area: use of local vegetables; coastal area: seafood-centered), and stress management methods (e.g., urban area: meditation, gym use; rural area: nature walks; coastal area: marine sports). Output examples include: (1) Exercise: park walking three times a week; diet: more vegetables when eating out; stress management: gym use; (2) Exercise: daily hiking; diet: local vegetables; stress management: nature walks; (3) Exercise: beach running twice a week; diet: seafood-centered; stress management: marine sports. These outputs are used for proposal screens and notification messages to users, enabling users to select and implement health plans optimized for their living environment. Internal processing of the AI model includes integration of geographic features and health examination values, environment-adaptive proposal generation, and learning using loss functions (cross-entropy loss, mean squared error). If AI is not used, similar processing is performed by rule-based proposal branching based on geographic conditions and living environment. The technical effect is that by utilizing AI, the proposal unit can integrally analyze the user's living environment, geographic conditions, and health condition in a high-dimensional space, and automatically generate realistic and highly feasible personalized proposals. Application fields include challenge proposal systems based on health examination results, lifestyle guidance for patients in medical institutions, corporate health management platforms, and municipal health policy promotion.

The reception unit can take into account the user's current mood and physical condition when receiving the user's health examination results. For example, if the user is tired, a simple input method is proposed. If the user is relaxed, detailed input is encouraged. Furthermore, the timing of input can be adjusted according to the user's mood and physical condition. Thus, the reception unit can provide a more comfortable data input experience by considering the user's mood and physical condition. Specifically, the reception unit obtains the user's mood and physical condition data (e.g., self-reported questionnaire, biometric indicators from wearable devices, facial images, voice data, etc.). The questionnaire is entered in a selection format such as “current mood: tired, normal, relaxed” or in a free description format. Biometric indicators such as heart rate, skin temperature, and activity level (numerical vectors) are obtained from wearable devices. Facial images (RGB image tensors, 128×128 pixels, 3 channels) and voice data (1D time series array, 16 kHz, 3-10 seconds) can also be used. The reception unit preprocesses these data (e.g., face region extraction, voice noise removal, questionnaire text normalization, standardization of biometric indicators), and inputs them into a multimodal AI model (e.g., integrated model of CNN for images, RNN for voice, MLP for biometric indicators, and Transformer for questionnaire). Examples of AI inputs include: (1) Questionnaire: tired, heart rate: 90 bpm, facial expression: neutral; (2) Questionnaire: relaxed, heart rate: 65 bpm, facial expression: smiling; (3) Questionnaire: normal, heart rate: 75 bpm, facial expression: frowning. The AI model generates outputs such as mood/physical condition label (e.g., fatigue, relaxation, normal), recommended input method (e.g., simple, detailed), and recommended input timing (e.g., immediate, later, night recommended). Output examples include: (1) Mood: fatigue, method: simple, timing: night; (2) Mood: relaxation, method: detailed, timing: immediate; (3) Mood: normal, method: normal, timing: later. These outputs are used for automatic switching of input methods on the reception screen, input timing control, and sending reminder notifications. Internal processing of the AI model includes feature extraction for each modality, concatenation of multidimensional feature vectors in the integration layer, fully connected layers for mood/physical condition classification, recommended method, and timing recommendation in multitask learning, and learning using loss functions (cross-entropy loss, mean squared error). If AI is not used, similar processing is performed by threshold determination of questionnaire answers and biometric indicators, and rule-based judgment of facial feature points. The technical effect is that by utilizing AI, the reception unit can estimate the user's mood and physical condition with high accuracy and in real time, and automatically optimize input methods and timing, thereby reducing input stress, improving input completion rate, optimizing user experience, and enhancing overall system efficiency. Application fields include health examination data reception systems, patient input support in medical institutions, corporate health management platforms, and municipal health management services.

The analysis unit can take into account the user's psychological state when analyzing the user's health examination results. For example, if the user is feeling stressed, advice on stress management is provided. If the user is relaxed, a specific action plan for maintaining health can also be proposed. Furthermore, the expression method of analysis results can be adjusted according to the user's psychological state. Thus, the analysis unit can achieve more effective health management by considering the user's psychological state. Specifically, the analysis unit integrates and inputs the user's psychological state data (e.g., self-reported stress questionnaire, facial images, voice data, biometric indicators from wearable devices, etc.) and health examination result data (e.g., numerical vectors such as blood pressure, heart rate, blood test values, etc.). The questionnaire is entered in a selection format such as “current stress level: high, medium, low” or in a free description format. Facial images (RGB image tensors, 128×128 pixels, 3 channels), voice data (1D time series array, 16 kHz, 3-10 seconds), and biometric indicators (heart rate variability, skin conductance, etc. as numerical vectors) can also be used. The analysis unit preprocesses these data (face region extraction, voice noise removal, questionnaire text normalization, standardization of biometric indicators), and inputs them into a multimodal AI model (integrated model of CNN for images, RNN for voice, MLP for biometric indicators, and Transformer for questionnaire). Examples of AI inputs include: (1) Questionnaire: high stress, heart rate variability: large, facial expression: frowning; (2) Questionnaire: relaxed, heart rate variability: small, facial expression: smiling; (3) Questionnaire: normal, heart rate variability: medium, facial expression: neutral. The AI model generates outputs such as psychological state label (e.g., stress, relaxation, normal), recommended advice (e.g., stress management method, action plan), and analysis result expression method (e.g., concise, detailed, visually emphasized). Output examples include: (1) State: stress, advice: deep breathing, meditation recommended, expression: concise; (2) State: relaxation, advice: exercise plan proposal, expression: detailed; (3) State: normal, advice: health maintenance, expression: normal. These outputs are transferred to the analysis result generation module and user health management screens, and analysis content and advice optimized for psychological state are automatically generated. Internal processing of the AI model includes feature extraction for each modality, psychological state classification, advice generation, recommendation of expression method in multitask learning, and learning using loss functions (cross-entropy loss, mean squared error). If AI is not used, similar processing is performed by threshold determination of questionnaire answers and biometric indicators, and rule-based judgment of facial feature points. The technical effect is that by utilizing AI, the analysis unit can integrally analyze the user's psychological state and health examination data in a high-dimensional space, and automatically generate individually optimized advice and analysis expressions. Application fields include health examination result analysis systems, patient explanation support in medical institutions, corporate health management platforms, and municipal health management services.

The proposal unit can take into account the user's social relationships when making proposals based on the user's health examination results. For example, if the user lives with family, a health plan that the whole family can work on together is proposed. If the user lives alone, a health plan that can be executed individually can also be proposed. Furthermore, health plans utilizing social support can be proposed based on the user's friendships and workplace environment. Thus, the proposal unit can make more feasible and effective proposals by considering the user's social relationships. Specifically, the proposal unit integrates and inputs the user's family composition data (e.g., number of cohabitants, family age composition, family health status), friendship data (e.g., friend list on SNS, participation status in health activity groups), workplace environment data (e.g., work style, presence or absence of workplace health initiatives), and health examination result data (e.g., physical fitness score, health risk label). The proposal unit preprocesses these data (one-hot encoding of family, friends, and workplace categories, standardization of health examination values, aggregation of group activity history), and inputs them into an AI model (e.g., social relationship integrated MLP plus group activity proposal generation model). Examples of AI inputs include: (1) Family: 4 people, all healthy; friends: participating in exercise group; workplace: health initiatives present; physical fitness score 80; (2) Living alone; friends: few; workplace: no health initiatives; physical fitness score 60; (3) Family: 2 people, living with elderly; friends: not participating in health group; workplace: telework; physical fitness score 70. The AI model generates outputs such as optimal health plan (e.g., family participation exercise, individual execution plan, friend/workplace collaboration plan), proposal for utilizing social support (e.g., group challenge, recommendation to participate in workplace health events), and feasibility score (0-1). Output examples include: (1) Plan: family weekend walking, support: family group challenge; (2) Plan: individual stretching, support: SNS reminder; (3) Plan: joint exercise with family and friends, support: participation in workplace event. These outputs are used for proposal screens and notification messages to users, enabling users to select and implement health plans optimized for their social relationships. Internal processing of the AI model includes integration of social relationship features, group activity proposal generation, feasibility score calculation, and learning using loss functions (cross-entropy loss, mean squared error). If AI is not used, similar processing is performed by rule-based proposal branching based on family composition and friendships. The technical effect is that by utilizing AI, the proposal unit can integrally analyze the user's social relationships and health condition in a high-dimensional space, and automatically generate personalized health plans with high feasibility and continuity. Application fields include challenge proposal systems based on health examination results, lifestyle guidance for patients in medical institutions, corporate health management platforms, and municipal health policy promotion.

The reception unit can take into account the user's past medical history when receiving the user's health examination results. For example, abnormal values can be detected by comparing current health examination results with the user's past treatment and surgery history. The user's past medical history can also be referenced to evaluate the risk of specific diseases. Furthermore, an automatic completion function can be provided to improve the accuracy of input data based on the user's medical history. Thus, the reception unit can utilize the user's past medical history to achieve more accurate and efficient data input. Specifically, the reception unit integrates and inputs, for each user, medical history data (e.g., treatment and surgery history, structured data such as diagnosis name, diagnosis date, treatment details, surgery date, past medical history labels), health examination result data (e.g., numerical vectors such as blood pressure, heart rate, blood test values), and past health examination input data. The reception unit preprocesses these data (categorization of treatment and surgery history, one-hot encoding of medical history labels, standardization of health examination values), and inputs them into an AI model (e.g., medical history integrated MLP plus AutoEncoder for anomaly detection plus regression model for completion value estimation). Examples of AI inputs include: (1) Treatment: hypertension treatment, 2019; surgery: none; medical history: hypertension; health examination value: blood pressure 140/90; (2) Treatment: diabetes treatment, 2020; surgery: knee surgery, 2018; medical history: diabetes; health examination value: blood glucose 130 mg/dL; (3) Treatment: none; surgery: appendectomy, 2015; medical history: none; health examination value: all normal. The AI model generates outputs such as anomaly detection labels (e.g., normal, caution, abnormal), disease risk evaluation (e.g., high risk of hypertension, medium risk of diabetes), and automatic completion candidate values (e.g., estimated blood pressure, blood glucose). Output examples include: (1) Anomaly: high blood pressure, caution; (2) Risk: medium diabetes, completion candidate: blood glucose 120 mg/dL; (3) Anomaly: none, completion candidate: all normal. These outputs are used for input assistance on the reception screen, anomaly warning display, automatic completion of input items, and sending reminder notifications. Internal processing of the AI model includes integration of medical history features, AutoEncoder for anomaly detection, regression model for completion value estimation, and learning using loss functions (cross-entropy loss, mean squared error). If AI is not used, similar processing is performed by rule-based anomaly determination and mean value completion based on medical history. The technical effect is that by utilizing AI, the reception unit can integrally analyze the user's medical history and health examination data in a high-dimensional space, and achieve improved accuracy of input data, automation of anomaly detection, efficiency of input operations, reduction of input errors, and enhancement of user experience. Application fields include health examination data reception systems, patient input support in medical institutions, corporate health management platforms, and municipal health management services.

The analysis unit can take into account the user's lifestyle habits when analyzing the user's health examination results. For example, the user's exercise habits and dietary habits can be used to evaluate health condition. The user's sleep patterns and stress levels can also be considered to evaluate health risks. Furthermore, specific improvement measures can be proposed based on the user's lifestyle habits. Thus, the analysis unit can achieve more personalized health management by considering the user's lifestyle habits. Specifically, the analysis unit integrates and inputs the user's exercise habit data (e.g., time series vectors of weekly exercise frequency, exercise type, exercise intensity), dietary habit data (e.g., daily nutrient intake, meal balance score), sleep pattern data (e.g., bedtime, wake-up time, sleep duration, sleep efficiency), stress level data (e.g., self-reported questionnaire, biometric indicators), and health examination result data (e.g., numerical vectors such as blood pressure, blood glucose, body weight). The analysis unit preprocesses these data (time series feature extraction, nutrition balance calculation, sleep efficiency scoring, stress indicator normalization), and inputs them into an AI model (e.g., lifestyle habit integrated MLP plus classification model for health risk evaluation plus generative model for improvement proposal). Examples of AI inputs include: (1) Exercise: walking twice a week; diet: lack of vegetables; sleep: 6 hours; stress: high; health examination value: blood pressure 140/90; (2) Exercise: running every day; diet: well balanced; sleep: 7.5 hours; stress: low; health examination value: all normal; (3) Exercise: almost none; diet: high fat; sleep: irregular; stress: medium; health examination value: weight gain. The AI model generates outputs such as health risk evaluation (e.g., high, medium, low), improvement proposal (e.g., increase exercise frequency, improve meal balance, review sleep habits, stress management method), and individual advice (e.g., recommend exercise three times a week, increase vegetable intake, fix bedtime). Output examples include: (1) Risk: high, improvement: increase exercise, increase vegetable intake; (2) Risk: low, improvement: maintain current status; (3) Risk: medium, improvement: review sleep habits. These outputs are transferred to the analysis result generation module and user health management screens, and personalized health advice and improvement proposals are automatically generated. Internal processing of the AI model includes integration of lifestyle habit features, health risk classification, improvement proposal generation, and learning using loss functions (cross-entropy loss, mean squared error). If AI is not used, similar processing is performed by rule-based judgment or simple scoring of lifestyle habit data. The technical effect is that by utilizing AI, the analysis unit can integrally analyze the user's lifestyle habits and health examination data in a high-dimensional space, and automatically generate individually optimized health risk evaluation and improvement proposals. Application fields include health examination result analysis systems, patient explanation support in medical institutions, corporate health management platforms, and municipal health management services.

The proposal unit can take into account the user's hobbies and interests when making proposals based on the user's health examination results. For example, if the user is interested in outdoor activities, proposals such as hiking or camping are made. If the user is interested in cooking, healthy recipes can also be proposed. Furthermore, plans that allow the user to maintain health while enjoying their hobbies and interests can be proposed. Thus, the proposal unit can make more attractive and feasible proposals by considering the user's hobbies and interests. Specifically, the proposal unit integrates and inputs the user's hobby and interest data (e.g., questionnaire responses, SNS post content, past behavior history as text data or category labels), health examination result data (e.g., physical fitness score, health risk label), and past challenge history. The proposal unit preprocesses these data (tokenization and vectorization of text, one-hot encoding of category labels, standardization of health examination values), and inputs them into an AI model (e.g., Transformer for hobby and interest feature extraction plus health status integrated MLP plus proposal generation model). Examples of AI inputs include: (1) Hobby: hiking, physical fitness score 80; (2) Hobby: cooking, low health risk; (3) Hobby: camping, physical fitness score 60. The AI model generates outputs such as optimal challenge proposals (e.g., hiking course proposal, healthy recipe proposal, camping event proposal), feasibility score (0-1), and enjoyment recommendation level (e.g., high, medium, low). Output examples include: (1) Proposal: weekend hiking, enjoyment: high; (2) Proposal: low-calorie recipe, enjoyment: medium; (3) Proposal: participate in camping event, enjoyment: high. These outputs are used for proposal screens and notification messages to users, enabling users to select and implement health plans optimized for their hobbies and interests. Internal processing of the AI model includes extraction of hobby and interest features, integration of health status, proposal generation, and learning using loss functions (cross-entropy loss, mean squared error). If AI is not used, similar processing is performed by rule-based proposal branching based on hobby and interest categories. The technical effect is that by utilizing AI, the proposal unit can integrally analyze the user's hobbies, interests, and health status in a high-dimensional space, and automatically generate attractive and highly feasible personalized proposals. Application fields include challenge proposal systems based on health examination results, lifestyle guidance for patients in medical institutions, corporate health management platforms, and municipal health policy promotion.

The reception unit can estimate the user's emotions when receiving the user's health examination results and provide input support based on the estimated emotions. For example, if the user feels anxious, a reassuring message is displayed. If the user is relaxed, detailed input is encouraged. Furthermore, an automatic completion function can be provided to reduce input effort according to the user's emotions. Thus, the reception unit can provide a more comfortable data input experience by considering the user's emotions. Specifically, the reception unit obtains facial images (RGB image tensors, 128×128 pixels, 3 channels), voice data (1D time series array, 16 kHz, 3-10 seconds), and questionnaire text (with category labels) for emotion estimation, and after preprocessing (face region extraction, voice noise removal, text normalization), inputs them into a multimodal AI model (integrated model of CNN for images, RNN for voice, and Transformer for text). Examples of AI inputs include: (1) Camera image: anxious face, voice: trembling, questionnaire: “feeling anxious”; (2) Camera image: smiling face, voice: calm tone, questionnaire: “relaxed”; (3) Camera image: neutral face, voice: monotone, questionnaire: “normal”. The AI model integrates these inputs and generates outputs such as emotion label (e.g., anxiety, relaxation, normal), input support content (e.g., display of reassuring message, encouragement of detailed input, recommendation of automatic completion). Output examples include: (1) Emotion: anxiety, support: display reassuring message, automatic completion; (2) Emotion: relaxation, support: encourage detailed input; (3) Emotion: normal, support: standard input. These outputs are used for input support display on the reception screen, automatic completion of input items, and sending reminder notifications. Internal processing of the AI model includes feature extraction for each modality, emotion classification, generation of input support content, and learning using loss functions (cross-entropy loss, mean squared error). If AI is not used, similar processing is performed by threshold determination of facial feature points and voice features, and rule-based judgment of questionnaire answers. The technical effect is that by utilizing AI, the reception unit can estimate the user's emotional state with high accuracy and in real time, and automatically optimize input support content, thereby reducing input stress, improving input completion rate, optimizing user experience, and enhancing overall system efficiency. Application fields include health examination data reception systems, patient input support in medical institutions, corporate health management platforms, and municipal health management services.

The following is a brief explanation of the processing flow of Example of the Embodiment. Specifically, the system is composed of three main modules: a reception unit, an analysis unit, and a proposal unit, and data is linked between each module to automate the entire process from receiving health examination data to analysis and challenge proposal to the user. The reception unit receives health examination results entered by the user (e.g., numerical vectors of blood pressure, heart rate, body weight, height, blood test values, each element being a real value, with a dimension of about 5 to 20) in real time and stores them in a database. The reception unit performs preprocessing of input data (e.g., missing value completion, anomaly detection, unit unification, category labeling) and transfers the data to the analysis unit. The analysis unit inputs the health examination results received from the reception unit into an AI model (e.g., multilayer perceptron, convolutional neural network, time series RNN) and analyzes and evaluates the user's physical fitness score, health risk, recommended exercise intensity, etc., in a high-dimensional space. Examples of AI inputs include: (1) Blood pressure: 120/80, heart rate: 70, body weight: 65 kg; (2) Blood glucose: 95 mg/dL, cholesterol: 180 mg/dL; (3) Physical fitness test score: 80, muscle strength score: 70. The AI model generates outputs such as physical fitness score (0-100), health risk label (low, medium, high), recommended exercise intensity (high, medium, low), and output examples include: (1) Physical fitness score: 85, health risk: low, recommended exercise intensity: high; (2) Physical fitness score: 60, health risk: medium, recommended exercise intensity: medium; (3) Physical fitness score: 40, health risk: high, recommended exercise intensity: low. These outputs are transferred to the proposal unit and used as basic data for challenge proposals to the user. The proposal unit integrates the health status evaluation results received from the analysis unit and the user's interests and concerns data (e.g., questionnaire responses, past behavior history, SNS post content as text data or category labels), and uses natural language processing models (e.g., BERT or Transformer series models) and multi-objective optimization algorithms (e.g., linear weighting, reinforcement learning-based recommendation models) to generate personalized challenge proposals for each user. Examples of AI inputs include: (1) Physical fitness score: 85, health risk: low, recommended exercise intensity: high, interest: running; (2) Physical fitness score: 60, health risk: medium, recommended exercise intensity: medium, interest: travel; (3) Physical fitness score: 40, health risk: high, recommended exercise intensity: low, interest: walking. The AI model outputs challenge proposal candidates (e.g., exercise type, difficulty, achievement goal, reason for recommendation as structured data) based on these input data, and output examples include: (1) Proposal: “You can challenge a full marathon”, difficulty: high, goal: complete 42.195 km, reason for recommendation: physical fitness score 85, interest: running; (2) Proposal: “You can challenge a specific tour during a trip to Machu Picchu”, difficulty: medium, goal: trekking at an altitude of 2500 m, reason for recommendation: physical fitness score 60, interest: travel; (3) Proposal: “You may be able to walk up to 10 km”, difficulty: low, goal: 10 km walking, reason for recommendation: physical fitness score 40, interest: walking. These outputs are used for user presentation screens and notification messages, enabling users to select challenges according to their health status and interests. Internal processing of the AI model includes weighting of input features, optimization of multiple objective functions, calculation of recommendation scores, branching of proposal content by threshold determination, and learning using loss functions (cross-entropy loss). If AI is not used, similar proposals are executed by conventional rule-based processing or simple category matching. The technical effect is that by utilizing AI in the reception unit, analysis unit, and proposal unit, the entire process from receiving health examination data to analysis and proposal can be automated with high accuracy and speed, greatly improving personalization, proposal accuracy, and user experience. Application fields include health examination result management systems, patient support platforms for medical institutions, corporate health management support, municipal health policy promotion, sports club member management, and personal health promotion applications.

Step 1: The reception unit receives the results of health examinations. The results of health examinations include blood pressure, heart rate, body weight, height, blood test results, and so on. The reception unit stores the health examination results entered by the user in a database. The reception unit can also receive health examination results in real time. Step 2: The analysis unit analyzes the health examination results received by the reception unit. The analysis unit evaluates the user's physical fitness and health condition using AI. For example, AI evaluates the user's cardiopulmonary function and muscle strength and proposes appropriate challenges. Step 3: The proposal unit proposes appropriate challenges to the user based on the analysis result obtained by the analysis unit. The proposal unit proposes specific challenges to the user using AI based on the user's interests and concerns. For example, proposals such as “You may be able to walk up to 10 km”, “You can challenge a full marathon”, “You can challenge a 10 km marathon”, and “You can challenge a specific tour during a trip to Machu Picchu” are made. Specifically, in Step 1, the system receives health examination results (e.g., numerical vectors such as blood pressure 120/80, heart rate 70, body weight 65 kg, height 170 cm, blood test values, each element being a real value, with a dimension of about 5 to 20) in real time through the reception unit and stores them in a database. The reception unit performs preprocessing of input data (e.g., missing value completion, anomaly detection, unit unification, category labeling) and transfers the data to the analysis unit. In Step 2, the analysis unit inputs the health examination results received from the reception unit into an AI model (e.g., multilayer perceptron, convolutional neural network, time series RNN) and analyzes and evaluates the user's physical fitness score, health risk, recommended exercise intensity, etc., in a high-dimensional space. Examples of AI inputs include: (1) Blood pressure: 120/80, heart rate: 70, body weight: 65 kg; (2) Blood glucose: 95 mg/dL, cholesterol: 180 mg/dL; (3) Physical fitness test score: 80, muscle strength score: 70. The AI model generates outputs such as physical fitness score (0-100), health risk label (low, medium, high), recommended exercise intensity (high, medium, low), and output examples include: (1) Physical fitness score: 85, health risk: low, recommended exercise intensity: high; (2) Physical fitness score: 60, health risk: medium, recommended exercise intensity: medium; (3) Physical fitness score: 40, health risk: high, recommended exercise intensity: low. These outputs are transferred to the proposal unit and used as basic data for challenge proposals to the user. In Step 3, the proposal unit integrates the health status evaluation results received from the analysis unit and the user's interests and concerns data (e.g., questionnaire responses, past behavior history, SNS post content as text data or category labels), and uses natural language processing models (e.g., BERT or Transformer series models) and multi-objective optimization algorithms (e.g., linear weighting, reinforcement learning-based recommendation models) to generate personalized challenge proposals for each user. Examples of AI inputs include: (1) Physical fitness score: 85, health risk: low, recommended exercise intensity: high, interest: running; (2) Physical fitness score: 60, health risk: medium, recommended exercise intensity: medium, interest: travel; (3) Physical fitness score: 40, health risk: high, recommended exercise intensity: low, interest: walking. The AI model outputs challenge proposal candidates (e.g., exercise type, difficulty, achievement goal, reason for recommendation as structured data) based on these input data, and output examples include: (1) Proposal: “You can challenge a full marathon”, difficulty: high, goal: complete 42.195 km, reason for recommendation: physical fitness score 85, interest: running; (2) Proposal: “You can challenge a specific tour during a trip to Machu Picchu”, difficulty: medium, goal: trekking at an altitude of 2500 m, reason for recommendation: physical fitness score 60, interest: travel; (3) Proposal: “You may be able to walk up to 10 km”, difficulty: low, goal: 10 km walking, reason for recommendation: physical fitness score 40, interest: walking. These outputs are used for user presentation screens and notification messages, enabling users to select challenges according to their health status and interests. Internal processing of the AI model includes weighting of input features, optimization of multiple objective functions, calculation of recommendation scores, branching of proposal content by threshold determination, and learning using loss functions (cross-entropy loss). If AI is not used, similar proposals are executed by conventional rule-based processing or simple category matching. The technical effect is that by utilizing AI in the reception unit, analysis unit, and proposal unit, the entire process from receiving health examination data to analysis and proposal can be automated with high accuracy and speed, greatly improving personalization, proposal accuracy, and user experience. Application fields include health examination result management systems, patient support platforms for medical institutions, corporate health management support, municipal health policy promotion, sports club member management, and personal health promotion applications.

The specific processing unit 290 sends the results of specific processing to the smart device 14. In the smart device 14, the control unit 46A causes the output device 40 to output the results of specific processing. The microphone 38B acquires voice indicating user input in response to the results of specific processing. The control unit 46A sends the voice data indicating user input acquired by the microphone 38B to the data processing device 12. In the data processing device 12, the specific processing unit 290 acquires the voice data.

The data generation model 58 is a so-called generative AI (Artificial Intelligence). An example of the data generation model 58 is a generative AI such as ChatGPT (registered trademark) (Internet search <URL: https://openai.com/blog/chatgpt>). The data generation model 58 is obtained by performing deep learning on a neural network. The data generation model 58 receives prompts containing instructions and inference data such as voice data indicating voice, text data indicating text, and image data indicating images (e.g., still image data or video data). The data generation model 58 performs inference according to the instructions indicated by the prompt on the input inference data and outputs the inference results in one or more data formats such as voice data, text data, or image data. The data generation model 58 includes, for example, text generation AI, image generation AI, and multimodal generation AI. Here, inference refers to, for example, analysis, classification, prediction, and/or summarization. The specific processing unit 290 performs the specific processing described above using the data generation model 58. The data generation model 58 may be a fine-tuned model that outputs inference results from prompts without instructions, and in this case, the data generation model 58 can output inference results from prompts without instructions. The data processing device 12 and the like may include multiple types of data generation models 58, and the data generation model 58 may include AI other than generative AI. AI other than generative AI may include, for example, linear regression, logistic regression, decision trees, random forests, support vector machines (SVM), k-means clustering, convolutional neural networks (CNN), recurrent neural networks (RNN), generative adversarial networks (GAN), or naive Bayes, among others, and can perform various processing but are not limited to such examples. Additionally, AI may be an AI agent. Furthermore, when processing is performed by AI in each part described above, the processing may be performed partially or entirely by AI but is not limited to such examples. Additionally, processing implemented by AI including generative AI may be replaced with rule-based processing, and rule-based processing may be replaced with processing implemented by AI including generative AI.

Moreover, the processing by the data processing system 10 described above is executed by the specific processing unit 290 of the data processing device 12 or the control unit 46A of the smart device 14, but it may be executed by both the specific processing unit 290 of the data processing device 12 and the control unit 46A of the smart device 14. Additionally, the specific processing unit 290 of the data processing device 12 acquires or collects necessary information for processing from the smart device 14 or external devices, and the smart device 14 acquires or collects necessary information for processing from the data processing device 12 or external devices.

Each of the plurality of elements including the aforementioned reception unit, analysis unit, and proposal unit is implemented by at least one of, for example, the smart device 14 and the data processing apparatus 12. For example, the reception unit is implemented by a control unit 46A of the smart device 14 and receives health examination results input by the user. The analysis unit is implemented, for example, by a specific processing unit 290 of the data processing apparatus 12 and evaluates the user's physical fitness and health condition using AI. The proposal unit is implemented, for example, by the specific processing unit 290 of the data processing apparatus 12 and proposes appropriate challenges to the user based on the analysis result. The correspondence between each unit and the apparatus or control unit is not limited to the examples described above and various modifications are possible.

### Second Embodiment

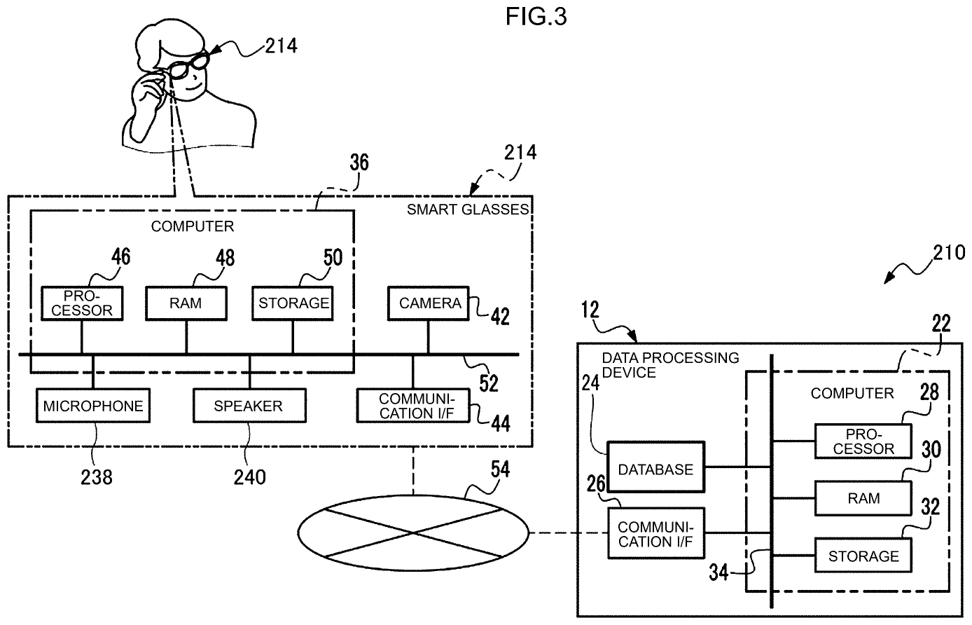

FIG. 3 shows an example configuration of a data processing system 210 according to the second embodiment.

As shown in FIG. 3, the data processing system 210 comprises a data processing device 12 and smart glasses 214. An example of the data processing device 12 is a server.

The data processing device 12 comprises a computer 22, a database 24, and a communication I/F 26. The computer 22 comprises a processor 28, RAM 30, and storage 32. The processor 28, RAM 30, and storage 32 are connected to a bus 34. Additionally, the database 24 and communication I/F 26 are also connected to the bus 34. The communication I/F 26 is connected to a network 54. Examples of the network 54 include a WAN and/or a LAN, among others.

The smart glasses 214 comprise a computer 36, a microphone 238, a speaker 240, a camera 42, and a communication I/F 44. The computer 36 comprises a processor 46, RAM 48, and storage 50. The processor 46, RAM 48, and storage 50 are connected to a bus 52. The microphone 238, speaker 240, and camera 42 are also connected to the bus 52.

The microphone 238 accepts voice from the user, accepting instructions, among others, from the user. The microphone 238 captures the voice emitted by the user, converts the captured voice into voice data, and outputs it to the processor 46. The speaker 240 outputs sound according to instructions from the processor 46.

The camera 42 is a small digital camera equipped with optical systems such as lenses, apertures, and shutters, as well as imaging elements such as CMOS (Complementary Metal-Oxide-Semiconductor) image sensors or CCD (Charge Coupled Device) image sensors, and captures the surroundings of the user (e.g., an imaging range defined by an angle of view equivalent to the typical field of view of a healthy person).

The communication I/F 44 is connected to the network 54. The communication I/F 44 and 26 manage the exchange of various information between the processor 46 and the processor 28 via the network 54. The exchange of various information between the processor 46 and the processor 28 using the communication I/F 44 and 26 is conducted securely.

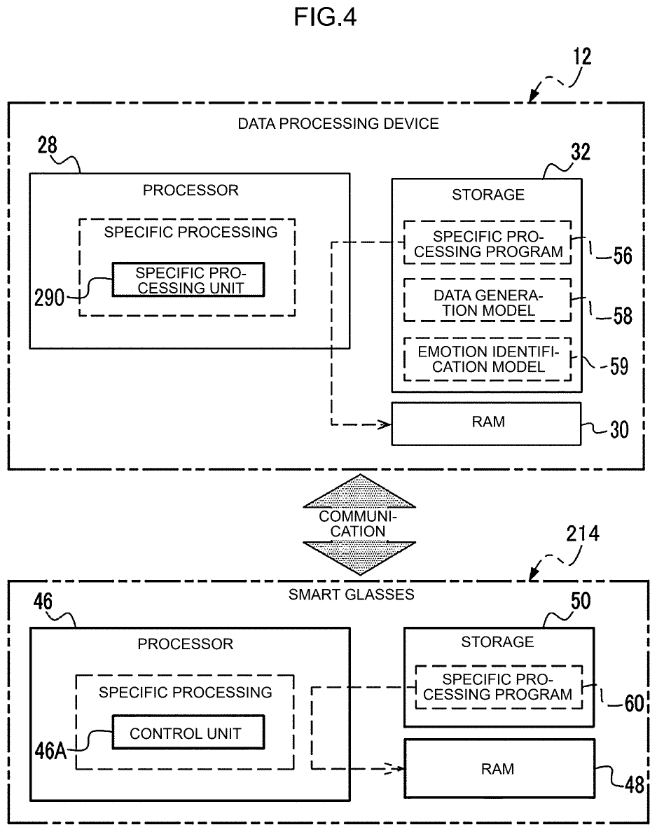

FIG. 4 shows an example of the main functions of the data processing device 12 and smart glasses 214. As shown in FIG. 4, specific processing is performed in the data processing device 12 by the processor 28. The storage 32 stores a specific processing program 56.

The processor 28 reads the specific processing program 56 from the storage 32 and executes it on the RAM 30. The specific processing is realized by the processor 28 operating as a specific processing unit 290 according to the specific processing program 56 executed on the RAM 30.

The storage 32 stores a data generation model 58 and an emotion identification model 59. The data generation model 58 and emotion identification model 59 are used by the specific processing unit 290. The specific processing unit 290 can estimate the user's emotions using the emotion identification model 59 and perform specific processing using the user's emotions. The emotion estimation function (emotion identification function) using the emotion identification model 59 includes estimating and predicting the user's emotions, but is not limited to such examples. Furthermore, emotion estimation and prediction may include, for example, emotion analysis.

In the smart glasses 214, specific processing is performed by the processor 46. The storage 50 stores a specific processing program 60. The processor 46 reads the specific processing program 60 from the storage 50 and executes it on the RAM 48. The specific processing is realized by the processor 46 operating as a control unit 46A according to the specific processing program 60 executed on the RAM 48. The smart glasses 214 may also have similar data generation models and emotion identification models as the data generation model 58 and emotion identification model 59, and perform the same processing as the specific processing unit 290 using these models.

Other devices besides the data processing device 12 may have the data generation model 58. For example, a server device may have the data generation model 58. In this case, the data processing device 12 communicates with the server device having the data generation model 58 to obtain processing results (e.g., prediction results) using the data generation model 58. The data processing device 12 may be a server device or a terminal device owned by the user (e.g., a mobile phone, robot, home appliance, etc.).

The specific processing unit 290 sends the results of specific processing to the smart glasses 214. In the smart glasses 214, the control unit 46A causes the speaker 240 to output the results of specific processing. The microphone 238 acquires voice indicating user input in response to the results of specific processing. The control unit 46A sends the voice data indicating user input acquired by the microphone 238 to the data processing device 12. In the data processing device 12, the specific processing unit 290 acquires the voice data.

The data generation model 58 is a so-called generative AI. An example of the data generation model 58 is a generative AI such as ChatGPT. The data generation model 58 is obtained by performing deep learning on a neural network. The data generation model 58 receives prompts containing instructions and inference data such as voice data indicating voice, text data indicating text, and image data indicating images (e.g., still image data or video data). The data generation model 58 performs inference according to the instructions indicated by the prompt on the input inference data and outputs the inference results in one or more data formats such as voice data, text data, or image data. The data generation model 58 includes, for example, text generation AI, image generation AI, and multimodal generation AI. Here, inference refers to, for example, analysis, classification, prediction, and/or summarization. The specific processing unit 290 performs the specific processing described above using the data generation model 58. The data generation model 58 may be a fine-tuned model that outputs inference results from prompts without instructions, and in this case, the data generation model 58 can output inference results from prompts without instructions. The data processing device 12 and the like may include multiple types of data generation models 58, and the data generation model 58 may include AI other than generative AI. AI other than generative AI may include, for example, linear regression, logistic regression, decision trees, random forests, support vector machines (SVM), k-means clustering, convolutional neural networks (CNN), recurrent neural networks (RNN), generative adversarial networks (GAN), or naive Bayes, among others, and can perform various processing but are not limited to such examples. Additionally, AI may be an AI agent. Furthermore, when processing is performed by AI in each part described above, the processing may be performed partially or entirely by AI but is not limited to such examples. Additionally, processing implemented by AI including generative AI may be replaced with rule-based processing, and rule-based processing may be replaced with processing implemented by AI including generative AI.

The data processing system 210 according to the second embodiment performs the same processing as the data processing system 10 according to the first embodiment. The processing by the data processing system 210 is executed by the specific processing unit 290 of the data processing device 12 or the control unit 46A of the smart glasses 214, but it may be executed by both the specific processing unit 290 of the data processing device 12 and the control unit 46A of the smart glasses 214. Additionally, the specific processing unit 290 of the data processing device 12 acquires or collects necessary information for processing from the smart glasses 214 or external devices, and the smart glasses 214 acquires or collects necessary information for processing from the data processing device 12 or external devices.

Each of the plurality of elements including the aforementioned reception unit, analysis unit, and proposal unit is implemented by at least one of, for example, the smart glasses 214 and the data processing apparatus 12. For example, the reception unit is implemented by a control unit 46A of the smart glasses 214 and receives health examination results input by the user. The analysis unit is implemented, for example, by a specific processing unit 290 of the data processing apparatus 12 and evaluates the user's physical fitness and health condition using AI. The proposal unit is implemented, for example, by the specific processing unit 290 of the data processing apparatus 12 and proposes appropriate challenges to the user based on the analysis result. The correspondence between each unit and the apparatus or control unit is not limited to the examples described above and various modifications are possible.

### Third Embodiment

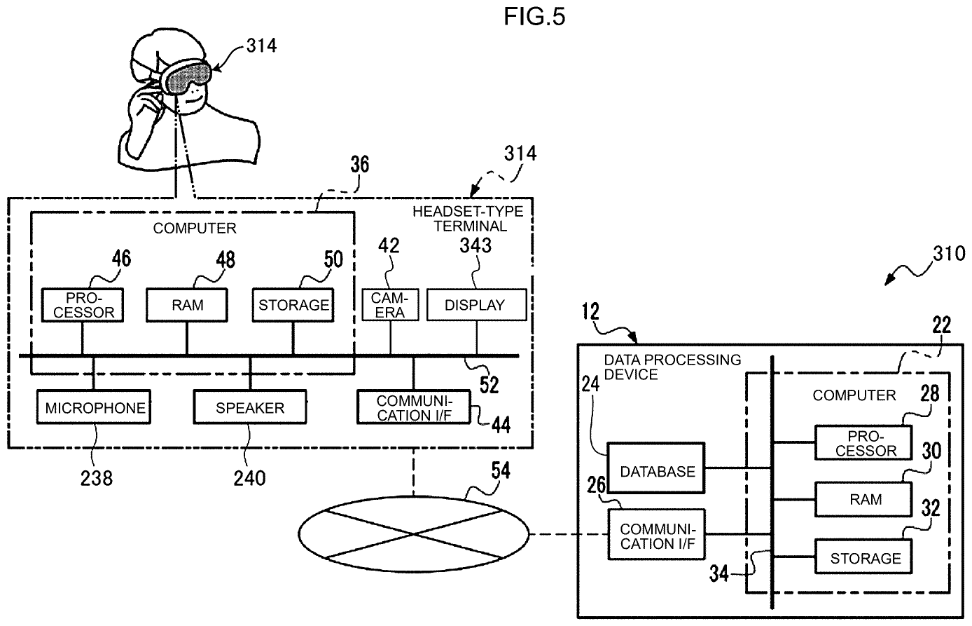

FIG. 5 shows an example configuration of a data processing system 310 according to the third embodiment.

As shown in FIG. 5, the data processing system 310 comprises a data processing device 12 and a headset-type terminal 314. An example of the data processing device 12 is a server.

The data processing device 12 comprises a computer 22, a database 24, and a communication I/F 26. The computer 22 comprises a processor 28, RAM 30, and storage 32. The processor 28, RAM 30, and storage 32 are connected to a bus 34. Additionally, the database 24 and communication I/F 26 are also connected to the bus 34. The communication I/F 26 is connected to a network 54. Examples of the network 54 include a WAN and/or a LAN, among others.

The headset-type terminal 314 comprises a computer 36, a microphone 238, a speaker 240, a camera 42, a communication I/F 44, and a display 343. The computer 36 comprises a processor 46, RAM 48, and storage 50. The processor 46, RAM 48, and storage 50 are connected to a bus 52. The microphone 238, speaker 240, camera 42, and display 343 are also connected to the bus 52.

The microphone 238 accepts voice from the user, accepting instructions, among others, from the user. The microphone 238 captures the voice emitted by the user, converts the captured voice into voice data, and outputs it to the processor 46. The speaker 240 outputs sound according to instructions from the processor 46.

The camera 42 is a small digital camera equipped with optical systems such as lenses, apertures, and shutters, as well as imaging elements such as CMOS (Complementary Metal-Oxide-Semiconductor) image sensors or CCD (Charge Coupled Device) image sensors, and captures the surroundings of the user (e.g., an imaging range defined by an angle of view equivalent to the typical field of view of a healthy person).

The communication I/F 44 is connected to the network 54. The communication I/F 44 and 26 manage the exchange of various information between the processor 46 and the processor 28 via the network 54. The exchange of various information between the processor 46 and the processor 28 using the communication I/F 44 and 26 is conducted securely.

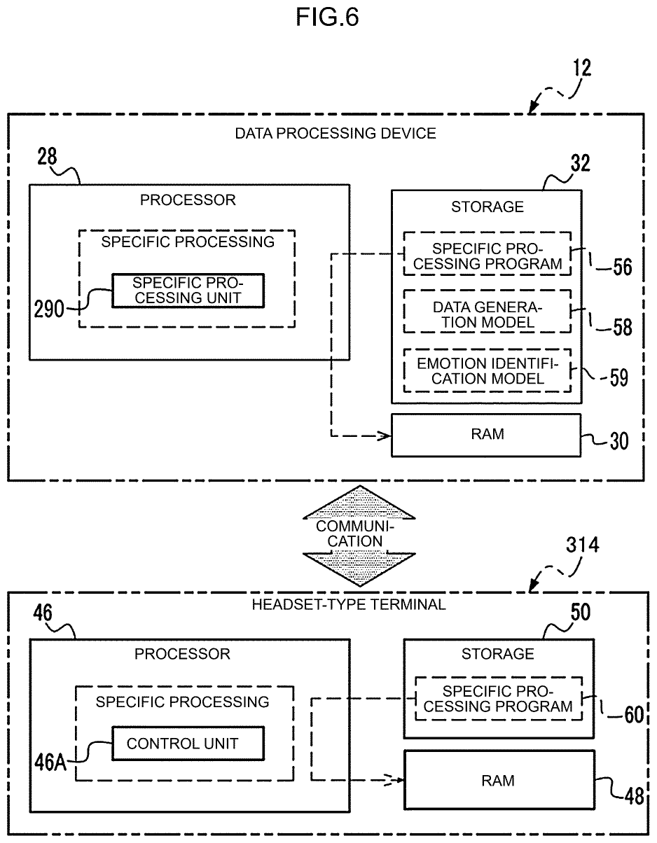

FIG. 6 shows an example of the main functions of the data processing device 12 and the headset-type terminal 314. As shown in FIG. 6, specific processing is performed in the data processing device 12 by the processor 28. The storage 32 stores a specific processing program 56.

The processor 28 reads the specific processing program 56 from the storage 32 and executes it on the RAM 30. The specific processing is realized by the processor 28 operating as a specific processing unit 290 according to the specific processing program 56 executed on the RAM 30.

The storage 32 stores a data generation model 58 and an emotion identification model 59. The data generation model 58 and emotion identification model 59 are used by the specific processing unit 290. The specific processing unit 290 can estimate the user's emotions using the emotion identification model 59 and perform specific processing using the user's emotions. The emotion estimation function (emotion identification function) using the emotion identification model 59 includes estimating and predicting the user's emotions, but is not limited to such examples. Furthermore, emotion estimation and prediction may include, for example, emotion analysis.

In the headset-type terminal 314, specific processing is performed by the processor 46. The storage 50 stores a specific program 60. The processor 46 reads the specific program 60 from the storage 50 and executes it on the RAM 48. The specific processing is realized by the processor 46 operating as a control unit 46A according to the specific program 60 executed on the RAM 48. The headset-type terminal 314 may also have similar data generation models and emotion identification models as the data generation model 58 and emotion identification model 59, and perform the same processing as the specific processing unit 290 using these models.

Other devices besides the data processing device 12 may have the data generation model 58. For example, a server device may have the data generation model 58. In this case, the data processing device 12 communicates with the server device having the data generation model 58 to obtain processing results (e.g., prediction results) using the data generation model 58. The data processing device 12 may be a server device or a terminal device owned by the user (e.g., a mobile phone, robot, home appliance, etc.).

The specific processing unit 290 sends the results of specific processing to the headset-type terminal 314. In the headset-type terminal 314, the control unit 46A causes the speaker 240 and the display 343 to output the results of specific processing. The microphone 238 acquires voice indicating user input in response to the results of specific processing. The control unit 46A sends the voice data indicating user input acquired by the microphone 238 to the data processing device 12. In the data processing device 12, the specific processing unit 290 acquires the voice data.

The data generation model 58 is a so-called generative AI. An example of the data generation model 58 is a generative AI such as ChatGPT. The data generation model 58 is obtained by performing deep learning on a neural network. The data generation model 58 receives prompts containing instructions and inference data such as voice data indicating voice, text data indicating text, and image data indicating images (e.g., still image data or video data). The data generation model 58 performs inference according to the instructions indicated by the prompt on the input inference data and outputs the inference results in one or more data formats such as voice data, text data, or image data. The data generation model 58 includes, for example, text generation AI, image generation AI, and multimodal generation AI. Here, inference refers to, for example, analysis, classification, prediction, and/or summarization. The specific processing unit 290 performs the specific processing described above using the data generation model 58. The data generation model 58 may be a fine-tuned model that outputs inference results from prompts without instructions, and in this case, the data generation model 58 can output inference results from prompts without instructions. The data processing device 12 and the like may include multiple types of data generation models 58, and the data generation model 58 may include AI other than generative AI. AI other than generative AI may include, for example, linear regression, logistic regression, decision trees, random forests, support vector machines (SVM), k-means clustering, convolutional neural networks (CNN), recurrent neural networks (RNN), generative adversarial networks (GAN), or naive Bayes, among others, and can perform various processing but are not limited to such examples. Additionally, AI may be an AI agent. Furthermore, when processing is performed by AI in each part described above, the processing may be performed partially or entirely by AI but is not limited to such examples. Additionally, processing implemented by AI including generative AI may be replaced with rule-based processing, and rule-based processing may be replaced with processing implemented by AI including generative AI.

The data processing system 310 according to the third embodiment performs the same processing as the data processing system 10 according to the first embodiment. The processing by the data processing system 310 is executed by the specific processing unit 290 of the data processing device 12 or the control unit 46A of the headset-type terminal 314, but it may be executed by both the specific processing unit 290 of the data processing device 12 and the control unit 46A of the headset-type terminal 314. Additionally, the specific processing unit 290 of the data processing device 12 acquires or collects necessary information for processing from the headset-type terminal 314 or external devices, and the headset-type terminal 314 acquires or collects necessary information for processing from the data processing device 12 or external devices.

Each of the plurality of elements including the aforementioned reception unit, analysis unit, and proposal unit is implemented by at least one of, for example, the headset-type terminal 314 and the data processing apparatus 12. For example, the reception unit is implemented by a control unit 46A of the headset-type terminal 314 and receives health examination results input by the user. The analysis unit is implemented, for example, by a specific processing unit 290 of the data processing apparatus 12 and evaluates the user's physical fitness and health condition using AI. The proposal unit is implemented, for example, by the specific processing unit 290 of the data processing apparatus 12 and proposes appropriate challenges to the user based on the analysis result. The correspondence between each unit and the apparatus or control unit is not limited to the examples described above and various modifications are possible.

### Fourth Embodiment

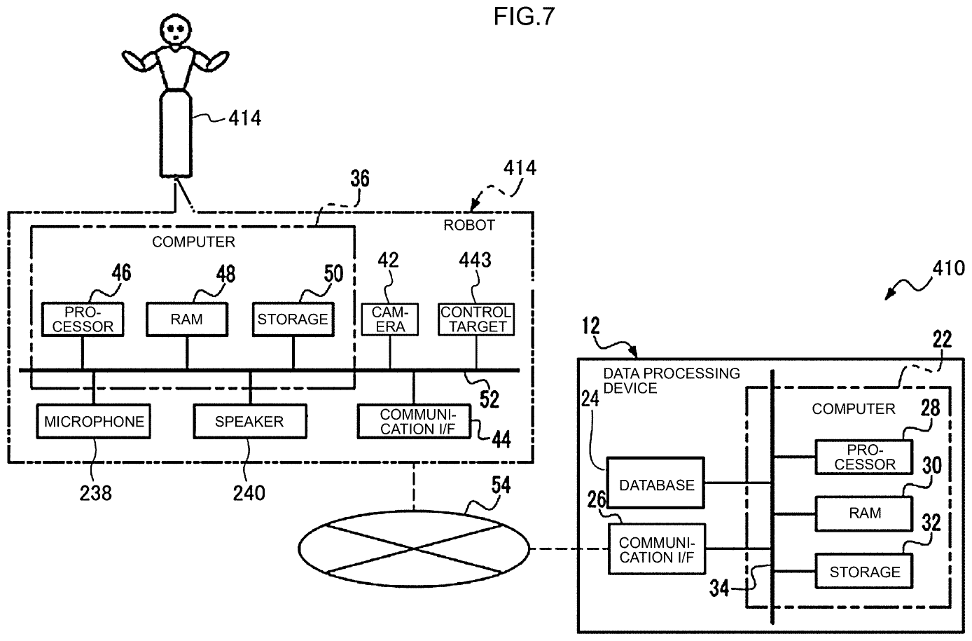

FIG. 7 shows an example configuration of a data processing system 410 according to the fourth embodiment.

As shown in FIG. 7, the data processing system 410 comprises a data processing device 12 and a robot 414. An example of the data processing device 12 is a server.

The data processing device 12 comprises a computer 22, a database 24, and a communication I/F 26. The computer 22 comprises a processor 28, RAM 30, and storage 32. The processor 28, RAM 30, and storage 32 are connected to a bus 34. Additionally, the database 24 and communication I/F 26 are also connected to the bus 34. The communication I/F 26 is connected to a network 54. Examples of the network 54 include a WAN and/or a LAN, among others.

The robot 414 comprises a computer 36, a microphone 238, a speaker 240, a camera 42, a communication I/F 44, and a control target 443. The computer 36 comprises a processor 46, RAM 48, and storage 50. The processor 46, RAM 48, and storage 50 are connected to a bus 52. The microphone 238, speaker 240, camera 42, and control target 443 are also connected to the bus 52.

The microphone 238 accepts voice from the user, accepting instructions, among others, from the user. The microphone 238 captures the voice emitted by the user, converts the captured voice into voice data, and outputs it to the processor 46. The speaker 240 outputs sound according to instructions from the processor 46.

The camera 42 is a small digital camera equipped with optical systems such as lenses, apertures, and shutters, as well as imaging elements such as CMOS image sensors or CCD image sensors, and captures the surroundings of the user (e.g., an imaging range defined by an angle of view equivalent to the typical field of view of a healthy person).

The communication I/F 44 is connected to the network 54. The communication I/F 44 and 26 manage the exchange of various information between the processor 46 and the processor 28 via the network 54. The exchange of various information between the processor 46 and the processor 28 using the communication I/F 44 and 26 is conducted securely.

The control target 443 includes a display device, LEDs for the eyes, and motors for driving arms, hands, and feet, among others. The posture and gestures of the robot 414 are controlled by controlling the motors for the arms, hands, and feet, among others. Some emotions of the robot 414 can be expressed by controlling these motors. Additionally, the expression of the robot 414 can be expressed by controlling the lighting state of the LEDs for the eyes of the robot 414.

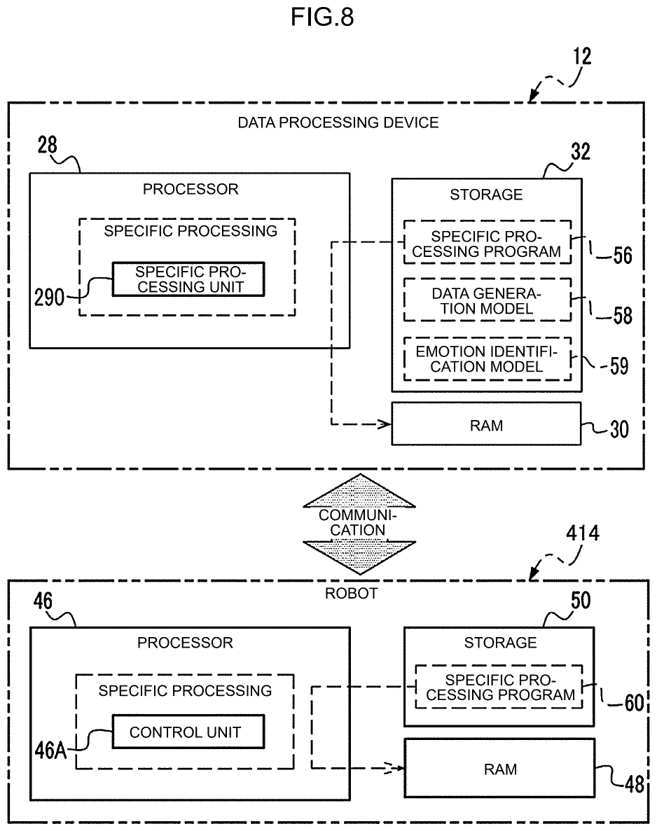

FIG. 8 shows an example of the main functions of the data processing device 12 and the robot 414. As shown in FIG. 8, specific processing is performed in the data processing device 12 by the processor 28. The storage 32 stores a specific processing program 56.

The processor 28 reads the specific processing program 56 from the storage 32 and executes it on the RAM 30. The specific processing is realized by the processor 28 operating as a specific processing unit 290 according to the specific processing program 56 executed on the RAM 30.

The storage 32 stores a data generation model 58 and an emotion identification model 59. The data generation model 58 and emotion identification model 59 are used by the specific processing unit 290. The specific processing unit 290 can estimate the user's emotions using the emotion identification model 59 and perform specific processing using the user's emotions. The emotion estimation function (emotion identification function) using the emotion identification model 59 includes estimating and predicting the user's emotions, but is not limited to such examples. Furthermore, emotion estimation and prediction may include, for example, emotion analysis.

In the robot 414, specific processing is performed by the processor 46. The storage 50 stores a specific program 60. The processor 46 reads the specific program 60 from the storage 50 and executes it on the RAM 48. The specific processing is realized by the processor 46 operating as a control unit 46A according to the specific program 60 executed on the RAM 48. The robot 414 may also have similar data generation models and emotion identification models as the data generation model 58 and emotion identification model 59, and perform the same processing as the specific processing unit 290 using these models.

Other devices besides the data processing device 12 may have the data generation model 58. For example, a server device may have the data generation model 58. In this case, the data processing device 12 communicates with the server device having the data generation model 58 to obtain processing results (e.g., prediction results) using the data generation model 58. The data processing device 12 may be a server device or a terminal device owned by the user (e.g., a mobile phone, robot, home appliance, etc.).

The specific processing unit 290 sends the results of specific processing to the robot 414. In the robot 414, the control unit 46A causes the speaker 240 and the control target 443 to output the results of specific processing. The microphone 238 acquires voice indicating user input in response to the results of specific processing. The control unit 46A sends the voice data indicating user input acquired by the microphone 238 to the data processing device 12. In the data processing device 12, the specific processing unit 290 acquires the voice data.

The data generation model 58 is a so-called generative AI. An example of the data generation model 58 is a generative AI such as ChatGPT. The data generation model 58 is obtained by performing deep learning on a neural network. The data generation model 58 receives prompts containing instructions and inference data such as voice data indicating voice, text data indicating text, and image data indicating images (e.g., still image data or video data). The data generation model 58 performs inference according to the instructions indicated by the prompt on the input inference data and outputs the inference results in one or more data formats such as voice data, text data, or image data. The data generation model 58 includes, for example, text generation AI, image generation AI, and multimodal generation AI. Here, inference refers to, for example, analysis, classification, prediction, and/or summarization. The specific processing unit 290 performs the specific processing described above using the data generation model 58. The data generation model 58 may be a fine-tuned model that outputs inference results from prompts without instructions, and in this case, the data generation model 58 can output inference results from prompts without instructions. The data processing device 12 and the like may include multiple types of data generation models 58, and the data generation model 58 may include AI other than generative AI. AI other than generative AI may include, for example, linear regression, logistic regression, decision trees, random forests, support vector machines (SVM), k-means clustering, convolutional neural networks (CNN), recurrent neural networks (RNN), generative adversarial networks (GAN), or naive Bayes, among others, and can perform various processing but are not limited to such examples. Additionally, AI may be an AI agent. Furthermore, when processing is performed by AI in each part described above, the processing may be performed partially or entirely by AI but is not limited to such examples. Additionally, processing implemented by AI including generative AI may be replaced with rule-based processing, and rule-based processing may be replaced with processing implemented by AI including generative AI.

The data processing system 410 according to the fourth embodiment performs the same processing as the data processing system 10 according to the first embodiment. The processing by the data processing system 410 is executed by the specific processing unit 290 of the data processing device 12 or the control unit 46A of the robot 414, but it may be executed by both the specific processing unit 290 of the data processing device 12 and the control unit 46A of the robot 414. Additionally, the specific processing unit 290 of the data processing device 12 acquires or collects necessary information for processing from the robot 414 or external devices, and the robot 414 acquires or collects necessary information for processing from the data processing device 12 or external devices.

Each of the plurality of elements including the aforementioned reception unit, analysis unit, and proposal unit is implemented by at least one of, for example, the robot 414 and the data processing apparatus 12. For example, the reception unit is implemented by a control unit 46A of the robot 414 and receives health examination results input by the user. The analysis unit is implemented, for example, by a specific processing unit 290 of the data processing apparatus 12 and evaluates the user's physical fitness and health condition using AI. The proposal unit is implemented, for example, by the specific processing unit 290 of the data processing apparatus 12 and proposes appropriate challenges to the user based on the analysis result. The correspondence between each unit and the apparatus or control unit is not limited to the examples described above and various modifications are possible.

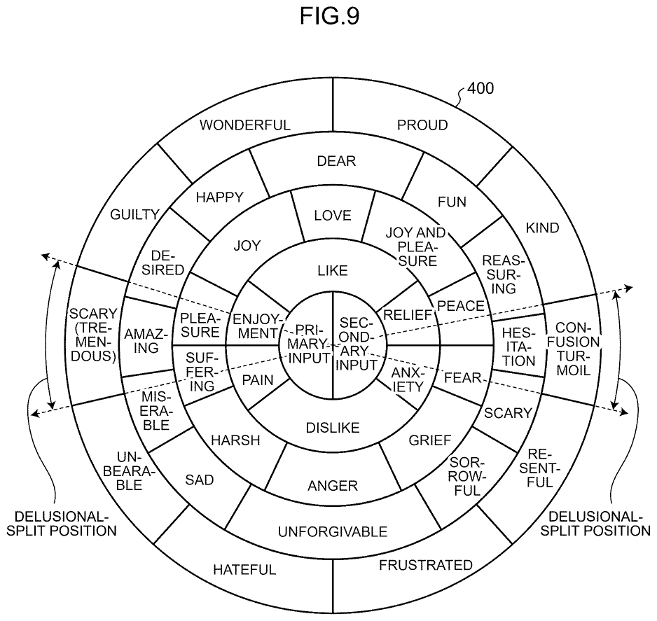

Note that the emotion identification model 59 as an emotion engine may determine the user's emotions according to a specific mapping. Specifically, the emotion identification model 59 may determine the user's emotions according to an emotion map, which is a specific mapping (see FIG. 9). Similarly, the emotion identification model 59 may determine the robot's emotions, and the specific processing unit 290 may perform specific processing using the robot's emotions.

FIG. 9 is a diagram showing an emotion map 400 where multiple emotions are mapped. In the emotion map 400, emotions are arranged concentrically radiating from the center. The closer to the center of the concentric circles, the more primitive the state of emotions is arranged. On the outer side of the concentric circles, emotions representing states and behaviors arising from mood are arranged. Emotions encompass concepts including emotional and mental states. On the left side of the concentric circles, emotions generally generated from reactions occurring in the brain are arranged. On the right side of the concentric circles, emotions generally induced by situational judgment are arranged. On the top and bottom of the concentric circles, emotions generated from reactions occurring in the brain and induced by situational judgment are arranged. Additionally, on the upper side of the concentric circles, “pleasant” emotions are arranged, and on the lower side, “unpleasant” emotions are arranged. In this way, in the emotion map 400, multiple emotions are mapped based on the structure from which emotions arise, and emotions that tend to occur simultaneously are mapped nearby.

These emotions are distributed in the 3 o'clock direction of the emotion map 400, and they usually move back and forth around reassurance and anxiety. In the right half of the emotion map 400, situational recognition takes precedence over internal sensations, giving a calm impression.

The inner side of the emotion map 400 represents the mind, and the outer side represents behavior, so the further out on the emotion map 400, the more visible (expressed in behavior) emotions become.

Here, human emotions are based on various balances like posture and blood sugar levels, and when these balances move away from the ideal, they indicate discomfort, and when they approach the ideal, they indicate comfort. In robots, cars, motorcycles, etc., emotions can be created based on various balances like posture and battery level, indicating discomfort when these balances move away from the ideal and comfort when they approach the ideal. The emotion map may be generated based on Dr. Mitsuyoshi's emotion map (Research on speech emotion recognition and brain physiological signal analysis systems related to emotions, Tokushima University, Doctoral dissertation: https://ci.nii.ac.jp/naid/500000375379). In the left half of the emotion map, emotions belonging to the domain called “reactions,” where sensations take precedence, are aligned. Additionally, in the right half of the emotion map, emotions belonging to the domain called “situations,” where situational recognition takes precedence, are aligned.

In the emotion map, two emotions that promote learning are defined. One is a negative emotion around “repentance” or “reflection” on the situation side. In other words, when a negative emotion arises in the robot, like “I never want to feel this way again” or “I don't want to be scolded again.” The other is an emotion around “desire” on the reaction side, which is positive. In other words, it is a positive feeling like “I want more” or “I want to know more.”

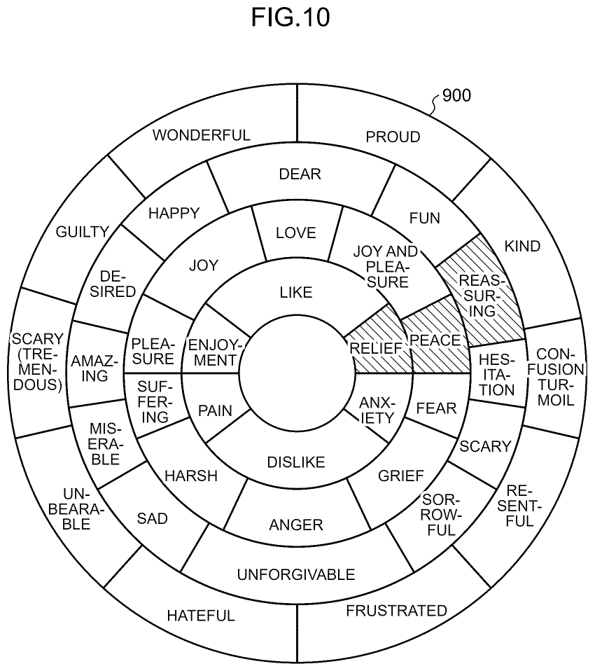

The emotion identification model 59 inputs user input into a pre-learned neural network, acquires emotion values indicating each emotion shown in the emotion map 400, and determines the user's emotions. This neural network is pre-learned based on multiple training data consisting of user input and combinations of emotion values indicating each emotion shown in the emotion map 400. Additionally, this neural network is learned so that emotions placed near each other in the emotion map 900 shown in FIG. 10 have similar values. FIG. 10 shows an example where multiple emotions like “reassured,” “calm,” and “confident” have similar emotion values.

In the above embodiments, an example form where specific processing is performed by a single computer 22 was described, but the technology disclosed herein is not limited to this, and distributed processing for specific processing by multiple computers including the computer 22 may be performed.

In the above embodiments, an example form where the specific processing program 56 is stored in the storage 32 was described, but the technology disclosed herein is not limited to this. For example, the specific processing program 56 may be stored in portable non-transitory storage media readable by a computer, such as a USB (Universal Serial Bus) memory. The specific processing program 56 stored in non-transitory storage media is installed in the computer 22 of the data processing device 12. The processor 28 executes specific processing according to the specific processing program 56.

Additionally, the specific processing program 56 may be stored in a storage device, such as a server connected to the data processing device 12 via the network 54, and downloaded and installed on the computer 22 in response to requests from the data processing device 12.

Furthermore, it is not necessary to store all of the specific processing program 56 in storage devices such as servers connected to the data processing device 12 via the network 54 or all in the storage 32, and a part of the specific processing program 56 may be stored.

Various processors, as shown next, can be used as hardware resources for executing specific processing. As processors, general-purpose processors that function as hardware resources for executing specific processing by executing software, i.e., programs, such as a CPU, can be mentioned. Additionally, as processors, dedicated electrical circuits with circuit configurations specially designed to execute specific processing, such as FPGA (Field-Programmable Gate Array), PLD (Programmable Logic Device), or ASIC (Application Specific Integrated Circuit), can be mentioned. Each processor has a built-in or connected memory, and each processor executes specific processing using the memory.

Hardware resources for executing specific processing may be composed of one of these various processors or a combination of two or more processors of the same or different types (e.g., a combination of multiple FPGAs or a combination of a CPU and FPGA). Additionally, hardware resources for executing specific processing may be a single processor.

As an example of composing with a single processor, firstly, there is a form where one or more CPUs and software are combined to constitute a single processor, which functions as hardware resources for executing specific processing. Secondly, there is a form using a processor, such as SoC (System-on-a-chip), that realizes the function of an entire system including multiple hardware resources for executing specific processing with a single IC chip. In this way, specific processing is realized using one or more of the various processors as hardware resources.

Furthermore, as a hardware structure of these various processors, more specifically, electrical circuits combined with circuit elements such as semiconductor elements can be used. Additionally, the specific processing described above is merely one example. Therefore, it goes without saying that unnecessary steps may be deleted, new steps may be added, or the order of processing may be changed within the scope not departing from the gist.

Additionally, in the examples described above, the explanation was divided into the first embodiment to the fourth embodiment, but parts or all of these embodiments may be combined. Additionally, the smart device 14, smart glasses 214, headset-type terminal 314, and robot 414 are examples, and each may be combined, or other devices may be used.

The descriptions and drawings shown above are detailed explanations of parts related to the technology disclosed herein and are merely examples of the technology disclosed herein. For example, the explanations regarding configurations, functions, actions, and effects above are explanations regarding examples of configurations, functions, actions, and effects of parts related to the technology disclosed herein. Therefore, it goes without saying that within the scope not departing from the gist of the technology disclosed herein, unnecessary parts may be deleted, new elements may be added, or replacements may be made to the descriptions and drawings shown above. Additionally, to avoid complexity and facilitate understanding of parts related to the technology disclosed herein, explanations concerning technical common knowledge and the like that do not require special explanation for enabling the implementation of the technology disclosed herein are omitted in the descriptions and drawings shown above.

All documents, patent applications, and technical standards described in this specification are incorporated by reference to the same extent as if each document, patent application, and technical standard were specifically and individually stated to be incorporated by reference in this specification.
(Supplementary Note 1) A system comprising: a reception unit configured to receive results of health examinations; an analysis unit configured to analyze the results of health examinations received by the reception unit; and a proposal unit configured to propose appropriate challenges to a user based on an analysis result obtained by the analysis unit.
(Supplementary Note 2) The system according to Supplementary Note 1, wherein the reception unit is configured to receive data of blood pressure, heart rate, body weight, height, and blood test results.
(Supplementary Note 3) The system according to Supplementary Note 1, wherein the analysis unit is configured to evaluate the user's physical fitness and health condition.
(Supplementary Note 4) The system according to Supplementary Note 1, wherein the proposal unit is configured to propose specific challenges based on the user's interests and concerns.
(Supplementary Note 5) The system according to Supplementary Note 1, wherein the proposal unit is configured to make proposals such as “able to walk up to 10 km,” “able to challenge a full marathon,” “able to challenge a 10 km marathon,” and “able to challenge a specific tour during a trip to Machu Picchu.”
(Supplementary Note 6) The system according to Supplementary Note 1, wherein the reception unit is configured to estimate the user's emotions and adjust the timing of inputting health examination results based on the estimated emotions of the user.
(Supplementary Note 7) The system according to Supplementary Note 1, wherein the reception unit is configured to analyze the user's past health examination results and select an appropriate input method.
(Supplementary Note 8) The system according to Supplementary Note 1, wherein the reception unit is configured to perform filtering based on the user's current lifestyle habits and dietary content when inputting health examination results.
(Supplementary Note 9) The system according to Supplementary Note 1, wherein the reception unit is configured to estimate the user's emotions and determine the priority of health examination results to be input based on the estimated emotions of the user.
(Supplementary Note 10) The system according to Supplementary Note 1, wherein the reception unit is configured to prioritize the input of highly relevant data based on the user's geographic location information when inputting health examination results.
(Supplementary Note 11) The system according to Supplementary Note 1, wherein the reception unit is configured to analyze the user's social media activities and input relevant data when inputting health examination results.
(Supplementary Note 12) The system according to Supplementary Note 1, wherein the analysis unit is configured to estimate the user's emotions and adjust the method of presenting analysis based on the estimated emotions of the user.
(Supplementary Note 13) The system according to Supplementary Note 1, wherein the analysis unit is configured to adjust the level of detail of analysis based on the importance of the health examination results during analysis.
(Supplementary Note 14) The system according to Supplementary Note 1, wherein the analysis unit is configured to apply different analysis algorithms according to the category of health examination results during analysis.
(Supplementary Note 15) The system according to Supplementary Note 1, wherein the analysis unit is configured to estimate the user's emotions and adjust the length of analysis based on the estimated emotions of the user.
(Supplementary Note 16) The system according to Supplementary Note 1, wherein the analysis unit is configured to determine the priority of analysis based on the submission timing of health examination results during analysis.
(Supplementary Note 17) The system according to Supplementary Note 1, wherein the analysis unit is configured to adjust the order of analysis based on the relevance of health examination results during analysis.
(Supplementary Note 18) The system according to Supplementary Note 1, wherein the proposal unit is configured to estimate the user's emotions and adjust the method of presenting proposals based on the estimated emotions of the user.
(Supplementary Note 19) The system according to Supplementary Note 1, wherein the proposal unit is configured to adjust the level of detail of proposals based on the importance of the challenges during proposal.
(Supplementary Note 20) The system according to Supplementary Note 1, wherein the proposal unit is configured to apply different proposal algorithms according to the category of the challenges during proposal.
(Supplementary Note 21) The system according to Supplementary Note 1, wherein the proposal unit is configured to estimate the user's emotions and adjust the length of proposals based on the estimated emotions of the user.
(Supplementary Note 22) The system according to Supplementary Note 1, wherein the proposal unit is configured to determine the priority of proposals based on the submission timing of the challenges during proposal.
(Supplementary Note 23) The system according to Supplementary Note 1, wherein the proposal unit is configured to adjust the order of proposals based on the relevance of the challenges during proposal.

What is claimed is:

1. A system comprising:
circuitry configured to:
receive, via a communication interface coupled to a packet-switched network, structured input data comprising multidimensional numerical vectors from a client terminal;
perform preprocessing on the structured input data comprising at least one of normalization or missing value imputation to generate preprocessed input data;
generate, using a data generation model comprising an encoder-decoder neural network obtained by performing deep learning on a neural network, inference output data based on the preprocessed input data, the encoder-decoder neural network receiving the preprocessed input data as multidimensional tensors and outputting the inference output data comprising a classification label and a numerical score; and
generate recommendation data based on the inference output data by applying at least one of threshold-based branching or a multi-objective optimization algorithm to the inference output data in combination with supplemental attribute data associated with a user.

2. The system according to claim 1, wherein the structured input data comprises at least five numerical parameters received as a real-valued vector with a dimensionality of 5 to 20.

3. The system according to claim 1, wherein the circuitry is further configured to evaluate a condition of the user based on the inference output data, the evaluation comprising generating the numerical score as a value in a range of 0 to 100 and the classification label as one of a plurality of predefined category labels.

4. The system according to claim 1, wherein the circuitry is further configured to generate the recommendation data based on the supplemental attribute data comprising at least one of interest data or preference data associated with the user, the interest data and the preference data being vectorized using a natural language processing model comprising a Transformer-based architecture.

5. The system according to claim 4, wherein the multi-objective optimization algorithm combines the inference output data with the vectorized interest data to generate personalized recommendation data for the user.

6. The system according to claim 1, wherein the circuitry is further configured to estimate an emotion of the user based on multimodal input data and to adjust a timing of receiving the structured input data based on the estimated emotion.

7. The system according to claim 6, wherein the multimodal input data comprises at least two of image data captured by a camera, voice waveform data captured by a microphone, or text data received via a touch panel, and wherein the circuitry estimates the emotion by inputting the multimodal input data into an emotion identification model comprising a fusion of a convolutional neural network for the image data, a recurrent neural network for the voice waveform data, and a Transformer for the text data.

8. The system according to claim 6, wherein the circuitry is further configured to, when the estimated emotion indicates stress, reduce a number of input fields presented on the client terminal for receiving the structured input data, and when the estimated emotion indicates relaxation, present an expanded number of input fields on the client terminal.

9. The system according to claim 1, wherein the circuitry is further configured to analyze past structured input data associated with the user stored in a database and to select an input method for the structured input data based on the past structured input data, the input method comprising at least one of automatic display of candidate input values or selection of a preferred input modality.

10. The system according to claim 1, wherein the circuitry is further configured to perform filtering on the structured input data based on supplemental contextual data associated with the user comprising at least one of lifestyle pattern data, dietary record data, or activity record data, and to prioritize input fields based on the filtering.

11. The system according to claim 1, wherein the circuitry is further configured to estimate an emotion of the user and to determine a priority order of the multidimensional numerical vectors to be received based on the estimated emotion, such that when the estimated emotion indicates stress, the circuitry prioritizes reception of numerical vectors associated with a high importance attribute.

12. The system according to claim 1, wherein the circuitry is further configured to select input fields for the structured input data based on geographic location information of the user received from the client terminal.

13. The system according to claim 1, wherein the circuitry is further configured to estimate an emotion of the user and to adjust a method of presenting the inference output data based on the estimated emotion, such that when the estimated emotion indicates stress, the circuitry generates the inference output data in a simplified format, and when the estimated emotion indicates relaxation, the circuitry generates the inference output data in a detailed format.

14. The system according to claim 1, wherein the circuitry is further configured to adjust a level of detail of the inference output data based on an importance score assigned to the structured input data, such that when the importance score exceeds a first threshold, the circuitry generates inference output data with increased granularity, and when the importance score is below a second threshold, the circuitry generates inference output data with reduced granularity.

15. The system according to claim 1, wherein the circuitry is further configured to apply different inference algorithms according to a category of the structured input data, the different inference algorithms comprising at least one of a convolutional neural network for pattern-based classification, a multilayer perceptron for score regression, or a Transformer-based model for sequential feature extraction.

16. The system according to claim 1, wherein the circuitry is further configured to estimate an emotion of the user and to adjust a method of presenting the recommendation data based on the estimated emotion, such that when the estimated emotion indicates stress, the circuitry generates the recommendation data in a concise format, and when the estimated emotion indicates relaxation, the circuitry generates the recommendation data in an expanded format.

17. The system according to claim 1, wherein the circuitry is further configured to determine a priority of the recommendation data based on a submission timing associated with the structured input data, such that when a submission timing is within a threshold period, the circuitry prioritizes generation of the recommendation data.

18. A system comprising:
a communication interface coupled to a packet-switched network;
a processor;
a random-access memory; and
a memory storing a data generation model and an emotion identification model, wherein the data generation model comprises an encoder-decoder neural network obtained by performing deep learning on a neural network, and the emotion identification model comprises a multimodal fusion model;
circuitry configured to:
receive, via the communication interface, structured input data comprising multidimensional numerical vectors from a client terminal;
perform preprocessing on the structured input data comprising normalization and missing value imputation to generate preprocessed input data;
generate, using the data generation model stored in the memory, inference output data by inputting the preprocessed input data as multidimensional tensors into the encoder-decoder neural network, the encoder-decoder neural network outputting the inference output data comprising a classification label and a numerical score;
generate recommendation data based on the inference output data by applying threshold-based branching and a multi-objective optimization algorithm to the inference output data in combination with supplemental attribute data associated with a user, the supplemental attribute data being vectorized using a natural language processing model; and
transmit, via the communication interface, the recommendation data to the client terminal.

19. The system according to claim 18, wherein the circuitry is further configured to estimate an emotion of a user by inputting multimodal input data comprising at least image data and voice waveform data into the emotion identification model stored in the memory, and to adjust at least one of a method of receiving the structured input data, a level of detail of the inference output data, or a display method of the recommendation data based on the estimated emotion.

20. A method performed by circuitry of a system, the method comprising:
receiving, via a communication interface coupled to a packet-switched network, structured input data comprising multidimensional numerical vectors from a client terminal;
performing preprocessing on the structured input data comprising at least one of normalization or missing value imputation to generate preprocessed input data;
generating, using a data generation model comprising an encoder-decoder neural network obtained by performing deep learning on a neural network, inference output data based on the preprocessed input data, the encoder-decoder neural network receiving the preprocessed input data as multidimensional tensors and outputting the inference output data comprising a classification label and a numerical score; and
generating recommendation data based on the inference output data by applying at least one of threshold-based branching or a multi-objective optimization algorithm to the inference output data in combination with supplemental attribute data associated with a user.
